SEQUENCE LISTING (2 sequences)

SEQ ID NO: 1 - DNA, 4956 bp, Enterococcus faecalis
ttattttcgtttcttcttgatgactataccactaataatcacaataagtaatacaacaagtcctagcacacgatatagaccattagtttgttctcccgttttgggataacgtccttgcttgtcaggtttcacaatgtgtgtgacatactgactactttttatcgtggtttgattcgttgaagaactagtctggttgttctcatttttatctggtagcggtgtaggattacgatcaatcacatgaacagttgctactgtcgttaattgacgaggtgtttcgtttgaaaacattgagaaaagtcgtgactgcacagctggtttggtttcattaactggctcagtggtataggaaatctggtactctcctgttttctttgtatcaacttccccttttacttgaaccttatcaaaggatatagcatgaccatcacgatctgtcgctgaaacgaaattatcttctggtttccacgaatcaccaacataaatcgttgtatctttaacctctaacttagattggtcgggtttgacggccacatgggctgtttcttctttaccttcgtaactgtatatgattggataaacgcctgctttgctagtatcaacttgacctgaaacagtgattttttcgaatgggacatcttgacctgttttatctgttgctgaaacaaagttctcttctggtttccatgaatcgccgacataaatcgttgtatccttgacttgtaaacgactatcatcacggacatgaacgattgctttcgcttcttttgtgccatttttatagacaatttcataatcgcctattttatcaacattcactgttccctgaacatcaattttttcaaacggaacgtcttgacctgttttgtctgtcgcggaaacgaagttatcttctgcttcccatttatcacccacataaattgttgtatctttgacctctaactgactgtcatcacggacatgaacgattgctttcgcttcttttgtgccatttttatagacaatttcataatcgcctattttatcaacattcactgttccctgaacgtcaattttttcaaacgggacatcttgacctgttttgtctgttgctgaaacgaaattatcttctggttcccatttatcgcccacataaatcgttgtatctttaacctctaacttagattggtcgggtttgacagtcacattagctgtttcttctttaccttcgtaactgtaaacgattggataaacgcctgctttgctagtatcaacttgacctgaaacagtgattttttcaaacggaacgtcttggcctgttttgtctgttgctgaaacgaaattatcttctggtttccacgaatcaccaacataaatcgttgtatctttaacctctaactgactgtcatcacggacatgaacgattgctttcgcttcttttttgccatttttatagacaatttcataatcgcctattttatcaacattcactgttccctgaacatcaattttttcaaacggaacgtcttgacctgttttgtctgtcgctgaaacgaaattatcttctggtttccacgaatcaccaacataaatcgttgtatctttgacctctaacttagattggtcgggtttgacggtcacataggctgtttcttctttaccttcgtcactgtaaataattggataaacgcctgctttggtgttatcaacttgacctgaaacagtgattttttcgaagggaacgtcttgacctgttttatctgttgctgaaacaaagttctcttctggtttccatgaatcgccgacataaatcgttgaatctttgacttgtaaacgactgtgatcaattgttttcgttaatggaatttttagttggttgtctcctttgaccagcttaatggcttttcctgtcaaatactcttcatccacggaatattcctgcggtacattcgttactcgcaaagtataatcacctgtcatgatggcatcaaaggagtattgacctttttcatctgttgtaaattcggttgctgtatgcgtgacattatcaattagttcaaaagaaaccttcgcaattgcttggttttcatcctctacgttatgcgtgtagaatgtggcagttgctaaataaacaataccttccgcatcaccttctgagaatttcaatgtgattggcgcagtggtgacttttttcttgctagcagacatctggttcaatgtttcagagtcgattgttgtttctgtatagatttggccagcgtttgcagagaccgtattgtattcaatcgtaatgttttctgtcaatgtatagtctttaaaaatgactttcgcaccgtttgtagtcttctctaatgaataagcatcttctggcacgcctttcacggtaatagaattcaaatcgatttgagcattggttgtgccttttggtgttaattcaatcgttggatttttcacacgatgtgttttattatcaatattttttgttgttactgtcgtttttgttacattttttaatgttggattcgccgcttgtgttgcactcaaaatgtctaacgcttcattttgaacagaaaccgaagcagagccttcattattattcgattgtggttctttcgctgtccctgttatataaagagttggcacgtcgatccagccattggcgtttttatactcaataatgtaacgtttgttggtttttccaaaatcaaacgtgtaactattggtattttcgtcaaaagtaatcgttggatatccagggctgtttggcgtcattggtttatcaaagtattgaccgcggtcccagtattgtggataaattgaatctacctgatcgtttgaaacatcataaatgtcaaaaagaacatcggcgccagctggaatttttgtacggacgcttagtgagtcaaaagaatcatttcgtgcattgacaaggaattgatttttactactttgaagtgttcgttcagaaccacgcttactattagtagttactgtcgtatcaactggagttaaaccttcagcactcattgttatcggtatcgtatcgactgggatacttttatcttttggcaaactatctggcacataattaaaaccaattggaatttggatttcgttggttattggcgtagtgaacttaataactgaaccatctgacgttggcgttactgtataatccgttcctaacttgtaataaaaataaatttcttttgtagctcgtaatgaaagataattaggatgctttactttaatttctaaattttgaatcggtttctctttgcttcccataactgagacagttgttcttgatgaataggcaccattttgatcataattaccccaagcagtttcagcattcaaaggattaacaacactaattggttcaatatattgatcggatactaattgaatggctttattaggaccaattacaggtgtacttaatgatagattaatgtccttcttgtcaaaatccattgttaaatcagaaatatttattcttaaatagctatcttttgtggccgttaaagtgagcttgccaccttcagttttaatatcaaaatagtcttccaattttacattttcatataaaggaccatacgccgtaaatttaggattcttaatttcttgaccgtttgtactctctaatacgatttgttgacccacttttgtttctgttaacgtgccataaattgttacataatatttccctttgctttgaaaactgatataggacatactacgcatttcacgatcagcaattactttacttgtttgactcgtcttagcagtaaaatcggttgtttgtttagttcctgtcgtacttttcaaagaaattgacccaatttttgttgttggatactgctgcgaatataagtagttatagtccgaagcactctctaaataaattgtccgattaatcgataaggaataggctttttgttgattcatgtttggaacagttacagcaatccgcccgagtgaattatcgcttaatgtataatctttgccaggtgttaacaaggtttgcgtttctggtaaaatttgaccattaatgtcgacatcgtaactgaaaacttgaggttccaagttatcttgttttccaaagacagcgcctggcgtctctgttgttaataattctaatgtttgattgccagttaacgtccggtctaaattataaaaaatttcgccatctaaccccgtcgttctggttcgttcgttatattgaattggttccacttgttcatataaatcaaacgagtaggtttttttattttttcctggagtttcaattttgattaatggttccgcctctgttgttagataactttttagttctactttaaaggaagtatctgaaactcgttgattaaacgtaatacttaatgttttggtcgccgcatcgactgcccaagttgcgccaatgcctgtcacttctccgctgacagttgaataacctaaatgttctggtaattgaactgtataaactgatccaggtgcataacctgtgtttccaacagcaaattggaacgtagcggttaccggtgttgtatttaattcgcggtgaacctctgtttggctgtattctgttccaccaatcggcgcaagactcatactgtcaagtagttcatctgtaattggcgcttgaacatctggcagggctttttcattaagtgaggtgtccttgtcctccgttgtttgttgttcagatggtgtagcagcatcactggaagctgttgtgctcgcctctgtggtttgcgattcagttgttgcagttggttgattcatttctgaagtagtcgttgttgctaaaggtaaagatggtgttgagttcgataactctgcctctttttttgctgtatcagtggtctctgtttgtgcttctgtcgttgttacagcataggcatttacggcaaacccgaaattttgtgcgagaagtatactcaacatcacaaaagaaaaagtttttttcttcat

SEQ ID NO: 2 - protein, 1651 aa, Enterococcus faecalis
MKKKTFSFVMLSILLAQNFGFAVNAYAVTTTEAQTETTDTAKKEAELSNSTPSLPLATTTTSEMNQPTATTESQTTEASTTASSDAATPSEQQTTEDKDTSLNEKALPDVQAPITDELLDSMSLAPIGGTEYSQTEVHRELNTTPVTATFQFAVGNTGYAPGSVYTVQLPEHLGYSTVSGEVTGIGATWAVDAATKTLSITFNQRVSDTSFKVELKSYLTTEAEPLIKIETPGKNKKTYSFDLYEQVEPIQYNERTRTTGLDGEIFYNLDRTLTGNQTLELLTTETPGAVFGKQDNLEPQVFSYDVDINGQILPETQTLLTPGKDYTLSDNSLGRIAVTVPNMNQQKAYSLSINRTIYLESASDYNYLYSQQYPTTKIGSISLKSTTGTKQTTDFTAKTSQTSKVIADREMRSMSYISFQSKGKYYVTIYGTLTETKVGQQIVLESTNGQEIKNPKFTAYGPLYENVKLEDYFDIKTEGGKLTLTATKDSYLRINISDLTMDFDKKDINLSLSTPVIGPNKAIQLVSDQYIEPISVVNPLNAETAWGNYDQNGAYSSRTTVSVMGSKEKPIQNLEIKVKHPNYLSLRATKEIYFYYKLGTDYTVTPTSDGSVIKFTTPITNEIQIPIGFNYVPDSLPKDKSIPVDTIPITMSAEGLTPVDTTVTTNSKRGSERTLQSSKNQFLVNARNDSFDSLSVRTKIPAGADVLFDIYDVSNDQVDSIYPQYWDRGQYFDKPMTPNSPGYPTITFDENTNSYTFDFGKTNKRYIIEYKNANGWIDVPTLYITGTAKEPQSNNNEGSASVSVQNEALDILSATQAANPTLKNVTKTTVTTKNIDNKTHRVKNPTIELTPKGTTNAQIDLNSITVKGVPEDAYSLEKTTNGAKVIFKDYTLTENITIEYNTVSANAGQIYTETTIDSETLNQMSASKKKVTTAPITLKFSEGDAEGIVYLATATFYTHNVEDENQAIAKVSFELIDNVTHTATEFTTDEKGQYSFDAIMTGDYTLRVTNVPQEYSVDEEYLTGKAIKLVKGDNQLKIPLTKTIDHSRLQVKDSTIYVGDSWKPEENFVSATDKTGQDVPFEKITVSGQVDNTKAGVYPIIYSDEGKEETAYVTVKPDQSKLEVKDTTIYVGDSWKPEDNFVSATDKTGQDVPFEKIDVQGTVNVDKIGDYEIVYKNGKKEAKAIVHVRDDSQLEVKDTTIYVGDSWKPEDNFVSATDKTGQDVPFEKITVSGQVDTSKAGVYPIVYSYEGKEETANVTVKPDQSKLEVKDTTIYVGDKWEPEDNFVSATDKTGQDVPFEKIDVQGTVNVDKIGDYEIVYKNGTKEAKAIVHVRDDSQLEVKDTTIYVGDKWEAEDNFVSATDKTGQDVPFEKIDVQGTVNVDKIGDYEIVYKNGTKEAKAIVHVRDDSRLQVKDTTIYVGDSWKPEENFVSATDKTGQDVPFEKITVSGQVDTSKAGVYPIIYSYEGKEETAHVAVKPDQSKLEVKDTTIYVGDSWKPEDNFVSATDRDGHAISFDKVQVKGEVDTKKTGEYQISYTTEPVNETKPAVQSRLFSMFSNETPRQLTTVATVHVIDRNPTPLPDKNENNQTSSSTNQTTIKSSQYVTHIVKPDKQGRYPKTGEQTNGLYRVLGLVVLLIVIISGIVIKKKRK

(19) United States
(12) Patent Application Publication
(10) Pub. No.: US 2011/0105383 A1
(43) Pub. Date: May 5, 2011

(54) METHODS AND COMPOSITIONS FOR STIMULATION OF MAMMALIAN INNATE IMMUNE RESISTANCE TO PATHOGENS

(76) Inventors: Magnus Hook, College Station, TX (US); Dekai Zhang, College Station, TX (US); Christi Gendron, College Station, TX (US); Burton Dickey, Houston, TX (US); Michael Tuvim, Houston, TX (US); Scott Evans, Bellaire, TX (US)

(21) Appl. No.: 12/556,759

(22) Filed: Sep. 10, 2009

Related U.S. Application Data
(60) Provisional application No. 61/156,254, filed on Feb. 27, 2009.
(60) Provisional application No. 61/191,570, filed on Sep. 10, 2008.

(51) Int. Cl. A61K 38/16; A61P 31/00; A61P 31/10; A61P 31/12; A61P 31/04; G01N 33/566; C07K 14/195; C12N 1/00

(52) U.S. Cl. 514/2.3; 514/2.4; 514/3.3; 514/3.7; 514/4.2; 514/3.4; 514/2.7; 514/21.2; 436/501; 530/350; 435/317.1

(57) ABSTRACT

Embodiments of the invention are directed to methods of treating, inhibiting or attenuating a microbial infection in an individual who has or is at risk for developing such an infection, comprising the step of administering an effective amount of a StIR polypeptide or peptide or fragment or derivative or analog thereof to the individual.

This application claims priority to U.S. Provisional Patent Application Ser. No. 61/156,254 filed Feb. 27, 2009 and Ser. No. 61/191,570 filed Sep. 10, 2008, each of which is incorporated herein by reference in its entirety.

### BACKGROUND OF THE INVENTION

I. Field of the Invention

The present invention relates generally to the fields of microbiology, immunology, and antimicrobial pharmacotherapy. More particularly the compositions and methods of the invention relate to modulation of innate immunity in the lungs of an individual for the treatment or attenuation of microbial infection or invasion.

II. Background

The susceptibility of the lungs to infection arises from the architectural requirements of gas exchange. To support ventilation, humans continuously expose 100 m² lung surface area to the external environment. Lungs are exposed not only to air, but also to the particles, droplets, and pathogens suspended in the air. Unlike cutaneous surfaces that are wrapped in impermeable skin or the gastrointestinal tract that is awash in a blanket of mucus, the lungs are the only significant environmental interface without an effective barrier defense. Such a barrier is precluded by the demand for unimpeded gaseous diffusion.

Despite their structural vulnerability, the lungs generally defend themselves successfully against infection through a variety of mechanical, humoral, and cellular mechanisms (Knowles et al., 2002; Martin and Frevert, 2005; Rogan et al., 2006; Travis et al., 2001). Most inhaled microbial pathogens fail to penetrate to the alveoli due to impaction against the airway walls, where they are entrapped by mucus and then expelled via the mucociliary escalator system (Knowles et al., 2002). For those pathogens that escape this fate, the constitutive presence of antimicrobial peptides in the airway lining fluid limits their growth (Rogan et al., 2006; Travis et al., 2001). Alveolar macrophages that reside in the most distal airspaces are able to ingest these organisms, thereby clearing the lungs from a potential infection.

Though often regarded as passive gas exchange barriers, the airway and alveolar epithelia supplement the baseline lung defenses by undergoing remarkable local structural and functional changes when pathogenic stimuli are encountered. In response to viral, fungal or allergic inflammation, airway secretory cells rapidly increase their height and fill their apical cytoplasm with secretory granules, a process termed inflammatory metaplasia (Evans et al., 2004; Williams et al., 2006). In the presence of pathogens, the alveolar epithelia activate their plasmalemmal systems and secretory machinery, thereby engaging leukocytes in lung protection (Evans et al., 2005). Perhaps most importantly, microbial interactions with respiratory epithelial pattern recognition receptors causes numerous microbicidal products to be expressed into the airway lining fluid, including defensins, cathelicidins, lysozyme and reactive oxygen species (Rogan et al., 2006; Forteza et al., 2005; Akinbi et al., 2000; Bals and Hiemstra, 2004; Bals and Hiemstra, 2006).

The 2001 anthrax attacks in the United States highlighted the challenges of defending populations against microbial infection via the respiratory system. Over 10,000 individuals required post-exposure prophylaxis; five of eleven individuals with confirmed pulmonary anthrax still died even though all received appropriate antimicrobial treatment (Schmitt et al., 2007; Bouzianas, 2007). Even if an adequate vaccine stockpile could be maintained for general distribution, vaccination in the midst of an event would not protect the population since protective vaccine administration occurs over months (Schmitt et al., 2007). Furthermore, vaccines are not available for all potential bioterror agents (Hassani et al., 2004). In the case that vaccines existed for all NIAID Class A pathogens, it would still be implausible to sufficiently stockpile doses for general distribution against each of them (Bouzianas, 2007; Hassani et al., 2004). Perhaps most importantly, the identity of a pathogen may not be immediately evident, delaying the determination of appropriate preventative and/or post-exposure therapies ((Schmitt et al., 2007). The broad protection conferred by stimulation of innate immunity is therefore highly attractive for management of large populations in the event of a bioterror attack.

There remains a need for additional methods and compositions for inhibiting and/or treating microbial infections.

### SUMMARY OF THE INVENTION

The present invention provides methods and compositions related to compounds that stimulate resistance to pathogens. In one aspect, the compound is a recombinant bacterial protein, (a Stimulated Innate Resistance (StIR) polypeptide).

In certain aspects, methods of treating, inhibiting or attenuating a microbial infection in an individual who has or is at risk for developing such an infection is contemplated, the methods comprising administering an effective amount of a StIR peptide, e.g. Enterococcus faecalis protein EF2505 (SEQ ID NO:2), or a fragment of derivative thereof to said individual. Typically, the individual or subject has been exposed to a pathogenic microbe or is at risk for such exposure. In certain aspects the StIR peptide is a purified or isolated polypeptide or peptide. The term “purified” or “isolated” means that component was previously isolated away or purified from other proteins and that the component is at least about 70, 75, 80, 90, 95, 97, or 99% pure prior to being formulated in the composition. In certain embodiments, the purified or isolated component is about or is at least about 95, 96, 97, 98, 99, 99.1, 99.2, 99.3, 99.4, 99.5% pure or more, or any range derivable therein. Such a purified component may then be mixed with other components to form a composition as described herein.

A recombinant StIR protein, e.g., EF2505, or fragment or segment thereof or analog thereof comprises at least, at most, or about 5, 10, 15, 20, 25, 30, 35, 40, 50, 55, 60, 65, 70, 75, 80, 85, 90, 95, 100, 150 200, 250, 300, 350, 400, 450, 500, 550, 600, 650, 700, 750, 800, 850, 900, 950, 1000, 1500, 1600 or 1651 consecutive amino acids, including all values and ranges there between, of SEQ ID NO:2. In certain aspects, a fragment or analog thereof includes at least or at most or about amino acid sequence from amino acid 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 45, 50, 55, 60, 65, 70, 75, 80, 85, 90, 95, 100, 110, 111, 112, 113, 114, 115, 116, 117, 118, 119, 120 to amino acid 100, 150, 200, 250, 300, 350, 355, 360, 365, 370, 375, 380, 390, 395, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450 of SEQ ID NO:2, including all values and ranges there between. In a further aspect, a polypeptide fragment or analog thereof includes, but is not limited to an amino acid sequence comprising at least, at most, or about amino acids 20, 21, 22, 23, 24, 25, 26, 27, 28, 29, 30 to amino acid 440, 441, 442, 443, 444, 445, 446, 447, 448, 449, 450 of SEQ ID NO:2. In certain aspects, a polypeptide segment or fragment or analog thereof includes, but is not limited to an amino acid sequence comprising at least or at most or about amino acids 28, 29, 30, 35, 40, 45, 50, 55, 60, 65, 70, 75, 80, 85, 90, 95, 100, 110, 120, 130, 140, 150, 200, 250, to amino acid 440, 441, 442, 443, 444, 445, 446, 447, 448, 449, 450 of SEQ ID NO:2, including all values and ranges there between. In yet a further aspect, a polypeptide fragment or analog thereof comprises an amino acid sequence comprising an amino acid sequence that is at least 70, 75, 80, 85, 90, 95, 96, 97, 98, 99 or 100% identical to amino acid 28 to 449, 28 to 442, 111 to 449, 111 to 442, 223 to 449, or 223 to 442 of SEQ ID NO:2, including all values and ranges there between. Derivatives or variants of the StIR protein or its segments includes insertion, deletion, and point mutations. A particular insertional mutation is a fusion protein that comprises amino acid sequence exogenous to the EF2505 protein at the carboxy or amino terminus.

In certain aspects, the StIR protein or a fragment or a segment or a derivative thereof is administered in a nebulized or aerosolized formulation. The composition can be administer by inhalation or inspiration. The StIR or a fragment of derivative thereof can be administered in an amount of from about 0.01, 0.05. 0.1, 0.5, 1, 5, 10, 15, 20, 25, 30, 35, 40, 45, 50, 55, 60, 65, 70 μg or mg/kg to about 55, 60, 65, 70, 75, 80, 85, 90, 95, 100, 125, 150, 200 μg or mg/kg of the individual's body weight. In other aspect, a subject can be administered about 0.01, 0.05. 0.1, 0.5, 1, 5, 10, 15, 20, 25, 30, 35, 40, 45, 50, 55, 60, 65, 70, 75, 80, 85, 90, 95, 100, 125, 150, 200 μg or mg or StIR polypeptide or peptide or variant or derivative or analog thereof. Based on the following disclosure, a person having ordinary skill in this art would readily be able to determine useful segments, fragments, or derivatives of a StIR polypeptide, e.g., Enterococcus faecalis protein EF2505. In one preferred aspect, the fragment, segment, or derivative is at least 75% identical to a sequence of SEQ ID NO:2 or a segment that corresponds to the claimed segment. In another aspect, the fragment, segment, or derivative is at least 80% identical to a sequence of SEQ ID NO:2 or a segment that corresponds to the claimed segment. In another aspect, the fragment, segment, or derivative is at least 85% identical to a sequence of SEQ ID NO:2 or a segment that corresponds to the claimed segment. In another aspect, the fragment, segment, or derivative is at least 90% identical to a sequence of SEQ ID NO:2 or a segment that corresponds to the claimed segment. In another aspect, the fragment, segment, or derivative is at least 95% identical to a sequence of SEQ ID NO:2 or a segment that corresponds to the claimed segment. A corresponding segment can be readily identified by visual inspection of the sequences or by computer alignment of SEQ ID NO:2 with the sequence of a claimed fragment.

In yet another embodiment, the present invention is directed to a pharmaceutically acceptable composition comprising one or more StIR polypeptide (e.g., Enterococcus faecalis protein EF2505) or a fragment or a segment or a derivative or an analog thereof; an anti-inflammatory agent; an anti-microbial agent; and/or one or more pharmaceutical excipients. Typically such compositions are sterile and essentially free of pathogenic microbes.

Embodiments of the invention include compositions, formulations, and methods for the enhancement of a mammalian, e.g., a human, subject's biological defenses against infection, for example the subject's immunity against infection. Aspects of the invention provide a rapid and temporal enhancement or augmentation of biological defenses against microbial infection. The enhancement of the immunity of a subject attenuates microbial infections. Attenuation can be by inhibiting, treating, or preventing infection or microbial growth or survival. Aspects of the invention enhance the defenses of the lung and respiratory tract of a subject.

In certain aspects the microbe is a virus, a bacteria, and/or a fungus. In certain aspects, a microbe is a virus. The virus can be from the Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, or Togaviridae family of viruses; and/or Parainfluenza, Influenza, H5N1, Marburg, Ebola, Severe acute respiratory syndrome coronavirus, Yellow fever virus, Human respiratory syncytial virus, Hantavirus, or Vaccinia virus.

In yet a further aspect, the pathogenic microbe is a bacteria. A bacteria can be an intracellular, a gram positive, or a gram negative bacteria. In a further aspect, the bacteria includes, but is not limited to a Staphylococcus, a Bacillus, a Francisella, or a Yersinia bacteria. In still a further aspect, the bacteria is Bacillus anthracis, Yersinia pestis, Francisella tularensis, Pseudomonas aerugenosa, or Staphylococcus aureas. In certain embodiments, a bacteria is Bacillus anthracis and/or Staphylococcus aureas. In still a further aspect, a bacteria is a drug resistant bacteria, such as a multiple drug resistant Staphylococcus aureas (MRSA). Representative medically relevant Gram-negative bacilli include Hemophilus influenzae, Klebsiella pneumoniae, Legionella pneumophila, Pseudomonas aeruginosa, Escherichia coli, Proteus mirabilis, Enterobacter cloacae, Serratia marcescens, and Helicobacter pylori, Salmonella enteritidis, and Salmonella typhi. Representative gram positive bacteria include but are not limited to Bacillus, Listeria, Staphylococcus, Streptococcus, Enterococcus, Actinobacteria and Clostridium Mycoplasma that lack cell walls and cannot be Gram stained, but are derived from such forms.

In still another aspect, the microbe is a fungus such as members of the family Aspergillus, Candida, Crytpococus, Histoplasma, Coccidioides, Blastomyces, Pneumocystis, or Zygomyces. In still further embodiments a fungus includes, but is not limited to Aspergillus fumigatus, Candida albicans, Cryptococcus neoformans, Histoplasma capsulatum, Coccidioides immitis, or Pneumocystis carinii. The family zygomycetes includes Basidiobolales (Basidiobolaceae), Dimargaritales (Dimargaritaceae), Endogonales (Endogonaceae), Entomophthorales (Ancylistaceae, Completoriaceae, Entomophthoraceae, Meristacraceae, Neozygitaceae), Kickxellales (Kickxellaceae), Mortierellales (Mortierellaceae), Mucorales, and Zoopagales. The family Aspergillus includes, but is not limited to Aspergillus caesiellus, A. candidus, A. carneus, A. clavatus, A. deflectus, A. flavus, A. fumigatus, A. glaucus, A. nidulans, A. niger, A. ochraceus, A. oryzae, A. parasiticus, A. penicilloides, A. restrictus, A. sojae, A. sydowi, A. tamari, A. terreus, A. ustus, A. versicolor, and the like. The family Candida includes, but is not limited to Candida albicans, C. dubliniensis, C. glabrata, C. guilliermondii, C. kefyr, C. krusei, C. lusitaniae, C. milleri, C. oleophila, C. parapsilosis, C. tropicalis, C. utilis, and the like.

Embodiments of the invention can be administered via the respiratory tract. In certain aspects administration is by inhalation. In a further aspect the composition is aerosolized or nebulized or in a form that can be inhaled by or instilled in a subject.

Methods of the invention include the administration of a composition by inhalation or other methods of administration to the upper and/or lower respiratory tract. In certain aspects the composition is aerosolized or aspirated. The subject can be at risk of exposure to or exposed to an airborne virus, bacteria, or fungus. In certain aspects the pathogenic bacteria is an intracellular, a gram positive, or a gram negative bacterium. In certain embodiments the bacteria is a Streptococcus, Staphylococcus, Bacillus, Francisella, or Yersinia. In still further aspects the bacteria is Bacillus anthracis, Yersinia pestis, Francisella tularensis, Streptococcus pnemoniae, Staphylococcus aureas, Pseudomonas aeruginosa, and/or Burkholderia cepacia.

Still further embodiments include methods where the composition is administered before; after; during; before and after; before and during; during and after; before, after and during exposure or suspected exposure or heightened risk of exposure to the organism. The subject can be exposed to a bioweapon or to an opportunistic pathogen. In particular aspects the subject is immunocompromised, such as a cancer patient or an AIDS patient.

In further embodiments, an anti-viral agent or composition is administered before, during, and/or after administration of a StIR protein or peptide. An anti-viral composition can comprise an immunoglobulin, a fusion inhibitor, an uncoating inhibitor, an interferon, a nucleotide analog, and/or a protease inhibitor. In certain aspects an anti-viral agent is a nucleotide analog, including, but not limited to ribivirin, vidarabine, acyclovir, gangcyclovir, zidovudine, didanosine, zalcitabine, stavudine, or lamivudine. In certain aspects of the invention the nucleotide analog is ribivirin, e.g., megaribivirin (i.e., a composition comprising ribivirin at a concentration of at least, at most, or about 5, 25, 50, 75 or 75, 100, 125, 250 mg/ml, including all values and ranges there between). An anti-viral agent can be administered at a dose of at least, at most, or about 0.1, 1, 5, 10, 20, 50, 100, 1000 ng, μg, or mg per kg of subject body weight. The microbial lysate and/or anti-viral agent/composition can be administered to the subject at least, at most, or about 1, 2, 3, 4, 5, 6, 7, 8, 9, 10 or more times. In certain aspects the subject is immunocompromised, e.g., infected with an immunodeficiency virus.

Certain embodiments also include pharmaceutically acceptable compositions comprising a microbial lysate, an anti-viral agent, and one or more pharmaceutical excipients, wherein said composition is sterile and essentially free of pathogenic microbes. In certain aspects the antiviral agent is present in a concentration of 0.1, 1, 15, 10, 20, 30, 40, 50, 60, 70, 80, 90, 100, 500, or 1000 ng, □g, or mg/ml, including all values and range there between.

One embodiment of the invention is directed to a method of identifying compounds that stimulate mammalian innate immune resistance to pathogens, comprising the steps of measuring the binding of a compound to leucine-rich repeat containing proteins to determine a compound which exhibits binding; and measuring the ability of compounds which bind to leucine-rich repeat containing proteins to activate the mammalian immune system, wherein a compound that binds to leucine-rich repeat containing proteins and activate the mammalian immune system is a compound that stimulates mammalian innate immune resistance to pathogens.

In yet another embodiment, the present invention is directed to a compound that binds to leucine-rich repeat containing proteins and activates the mammalian immune system to stimulate mammalian innate immune resistance to pathogens, wherein said compound is identified using the method described herein.

Other aspects of the invention include the ability to readily produce in large quantities of the inventive compositions.

The terms “attenuating,” “inhibiting,” “reducing,” or “prevention,” or any variation of these terms, when used in the claims and/or the specification includes any measurable decrease or complete inhibition to achieve a desired result, e.g., reduction in post-exposure bacterial load or growth.

The use of the word “a” or “an” when used in conjunction with the term “comprising” in the claims and/or the specification may mean “one,” but it is also consistent with the meaning of “one or more,” “at least one,” and “one or more than one.”

It is contemplated that any embodiment discussed herein can be implemented with respect to any method or composition of the invention, and vice versa. Furthermore, compositions and kits of the invention can be used to achieve methods of the invention.

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows that endogenous EF2505 increases the survival of mice exposed to S. pneumoniae inhalation.

FIG. 2 shows that EF2505 activates NF-kB in mammalian cells.

FIG. 3 shows that NF-kB activation is not due to DNA, RNA or LPS contamination in the purified EF2505 preparation.

FIG. 4 shows that EF2505 and NTHi stimulation of primary peritoneal macrophages is MyD88 dependent.

FIG. 5 shows that purified EF2505 increases the survival of mice exposed to P. aeruginosa and A. fumigatus challenge.

FIG. 6 shows a domain diagram of full-length EF2505 protein.

### DETAILED DESCRIPTION OF THE INVENTION

The immune system is the system of specialized cells and organs that protect an organism from outside biological influences. When the immune system is functioning properly, it protects the body against bacteria and viral infections, destroying cancer cells and foreign substances. If the immune system weakens, its ability to defend the body also weakens, allowing pathogens to grow in the body.

The immune system is often divided into: (a) an innate immunity comprised of components that provide an immediate “first-line” of defense to continuously ward off pathogens and (b) an adaptive (acquired) immunity comprising the manufacture of antibodies and production or stimulation of T-cells specifically designed to target particular pathogens. Using adaptive immunity the body can develop over time a specific immunity to particular pathogen(s). This response takes days to develop, and so is not effective at preventing an initial invasion, but it will normally prevent any subsequent infection, and also aids in clearing up longer-lasting infections.

In response to certain inflammatory stimuli, the secretory cells of the airway epithelium of mice and humans rapidly undergo a remarkable change in structure termed inflammatory metaplasia. Most of the structural changes can be ascribed to increased production of secreted, gel-forming mucins, while additional macromolecules with functions in mucin secretion, microbial killing or inflammatory signaling are also upregulated. The physiologic function of this response is thought to be augmentation of local defenses against microbial pathogens, although that hypothesis has received only limited formal testing. Paradoxically, excessive production and secretion of gel-forming mucins is a major cause of airflow obstruction in common inflammatory diseases of the airways such as asthma, cystic fibrosis, and chronic obstructive pulmonary disease (COPD). The stimulation of the innate immunity without the production of mucin would provide an additional method of attenuating infection of the respiratory tract by preventing and/or treating a subject.

Embodiments of the invention include the stimulation of the airways of a subject with a composition comprising a StIR protein (e.g., EF2505) or fragment or segment or derivative or analog thereof. A subject administered a composition of the invention is afford a therapeutic, prophylactic, or therapeutic and prophylactic response to a potentially infecting organism. In particular aspects, a composition is aerosolized and administered via the respiratory tract. The composition is used to induce or otherwise elicit a protective effect by, for example, activating or augmenting innate immunity of the lungs.

Embodiments of the invention include compositions comprising one or more StIR polypeptides or peptides. Aspects of the invention include StIR polypeptides or peptides derived from various microorganisms. Typically, the StIR polypeptide or peptide does not cause an increased production of secreted mucins. Embodiments of the invention can be used as a preventive and preemptive therapeutic against for example, bioweapons, neo-virulent microbes, or opportunistic microbes.

### I. POLYPEPTIDE AND PEPTIDE COMPOSITIONS

In certain embodiments, the present invention concerns at least one polypeptide or peptide or a derivative or variant thereof. As used herein, a “polypeptide,” “peptide,” “polypeptide or peptide composition,” or “polypeptide or peptide compound,” generally refers, but is not limited to, a protein or polypeptide of at least five amino acids or amino acid analogs (collectively an amino molecule, see below). All the “polypeptide or peptide” terms described above may be used interchangeably herein.

In certain embodiments the size of the at least one polypeptide or peptide molecule may comprise, but is not limited to, a molecule having about 5, 10, 50, 100, 500, 1000 to about 10, 50, 100, 500, 1000, 1500, 1651 or greater amino molecule residues, and any value or range derivable therein. The invention includes those lengths of contiguous amino acids or analogs thereof of any sequence discussed herein.

Segments or fragment of a polypeptide or peptide include amino acid 1, 2, 3, 4, 5, 6, 7, 8, 9, 10, 15, 20, 30, 40, 50, 60, 70, 80, 90, 100, 150, 200, 300, 350, 400, 450, 500, 550, 600, 650, 700, 750, 800, 850, 900, 950, 1000, 1500, 1600, 1610, 1620, 1630, 1640 to amino acid 10, 15, 20, 30, 40, 50, 60, 70, 80, 90, 100, 150, 200, 300, 350, 400, 450, 500, 550, 600, 650, 700, 750, 800, 850, 900, 950, 1000, 1500, 1600, 1651, including all values and ranges there between.

As used herein, an “amino molecule” refers to any amino acid, amino acid derivative or amino acid mimic as known to one of ordinary skill in the art. In certain embodiments, the residues of the polypeptide or peptide molecule are sequential, without any non-amino molecule interrupting the sequence of amino molecule residues. In other embodiments, the sequence may comprise one or more non-amino molecule moieties. In certain embodiments, the sequence of residues of the polypeptide or peptide molecule may be interrupted by one or more non-amino molecule moieties.

Accordingly, the term “polypeptide or peptide composition” encompasses amino molecule sequences comprising at least one of the 20 common amino acids in naturally synthesized proteins, or at least one modified or unusual amino acid.

In certain embodiments the polypeptide or peptide composition comprises at least one protein, polypeptide or peptide. In methods that involve an StIR polypeptide or peptide, compositions may comprise a polypeptide or peptide having all or part of the amino acid sequence of SEQ ID NO:2. In certain embodiments, protein, polypeptide, or peptide containing compositions will generally be proteins or peptides or synthetic proteins or peptides each essentially free from toxins, pathogens, and harmful immunogens.

Polypeptide or peptide compositions may be made by any technique known to those of skill in the art, including the expression of proteins, polypeptides or peptides through standard molecular biological techniques, the isolation of polypeptides or peptides from natural sources, or the chemical synthesis of polypeptide or peptide materials. The coding regions for these polypeptides or peptides may be amplified and/or expressed using the techniques disclosed herein or as would be know to those of ordinary skill in the art. Alternatively, various commercial preparations of proteins, polypeptides and peptides are known to those of skill in the art.

In certain embodiments a polypeptide or peptide compound may be purified. Generally, “purified” will refer to a specific or protein, polypeptide, or peptide composition that has been subjected to fractionation to remove various other proteins, polypeptides, peptides, and other molecules and compounds, and which composition substantially retains its activity, as may be assessed, for example, by protein assays, as known to one of ordinary skill in the art for the specific or desired protein, polypeptide or peptide.

It is contemplated that virtually any protein, polypeptide or peptide containing component may be used in the compositions and methods disclosed herein. In certain embodiments, it is envisioned that the formation of a aerosol or nebulized or aerosolizable or nebulizable composition will be advantageous in that will allow the composition to be more precisely or easily applied to the respiratory system by inhalation, inspiration, and the like.

### II. POLYPEPTIDE OR PEPTIDE VARIANTS AND DERIVATIVES

Amino acid sequence variants or derivatives of the proteins, polypeptides and peptides of the present invention can be substitutional, insertional or deletion variants. Deletion variants lack one or more residues of the native protein that are not essential for function or immunogenic activity. Another common type of deletion variant is one lacking secretory signal sequences or signal sequences directing a protein to bind to a particular part of a cell or membrane spanning regions or other functional sequences not needed for the in vivo activity sought. Insertional mutants typically involve the addition of material at a non-terminal point in the polypeptide. This may include the insertion of an immunoreactive epitope or simply a single residue. Terminal additions, called fusion proteins, are discussed below.

Substitutional variants typically contain the exchange of one amino acid for another at one or more sites within a polypeptide or peptide, and may be designed to modulate one or more properties, such as stability against proteolytic cleavage, without the loss of other functions or properties. Substitutions of this kind preferably are conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine.

The term “biologically functional equivalent” is well understood in the art and is further defined in detail herein. Accordingly, a biologically functional equivalent will have a sequence of about 70, 75, 80, 85, 90, 95, 96, 97, 98, 99% of amino acids that are identical or functionally equivalent to the amino acids of a polypeptide or peptide or variant or analog or derivative thereof and provide a similar biological activity to EF2505 or its segment or fragment or derivative or analog.

The following is a discussion based upon changing of the amino acids of a polypeptide or peptide to create an equivalent, or even an improved, second-generation molecule. For example, certain amino acids may be substituted for other amino acids in a polypeptide or peptide without appreciable loss of a particular activity such as, enhancement of immunologic response. Since it is the interactive capacity and nature of a polypeptide or peptide that typically defines a protein's functional activity, certain amino acid substitutions can be made in a polypeptide or peptide sequence, and in its underlying DNA coding sequence, and nevertheless produce a protein with like properties. It is thus contemplated by the inventors that various changes may be made in the DNA sequences encoding polypeptides or peptides of the invention without appreciable loss of their biological utility or activity, as discussed below.

In making such changes, the hydropathic index of amino acids may be considered. The importance of the hydropathic amino acid index in conferring interactive biologic function on a protein is generally understood in the art (Kyte & Doolittle, 1982). It is accepted that the relative hydropathic character of the amino acid contributes to the secondary structure of the resultant protein, which in turn defines the interaction of the protein with other molecules, cells, tissue and the like, for example, enzymes, substrates, receptors, DNA, antibodies, antigens, immunologic cells and systems, and the like.

It also is understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine *−0.5); cysteine (−1.0); methionine (−1.3); valine (−1.5); leucine (−1.8); isoleucine (−1.8); tyrosine (−2.3); phenylalanine (−2.5); tryptophan (−3.4).

It is also understood that an amino acid can be substituted for another having a similar hydrophilicity value and still produce a biologically equivalent and immunologically equivalent protein. In such changes, the substitution of amino acids whose hydrophilicity values are within ±2 is preferred, those that are within ±1 are particularly preferred, and those within ±0.5 are even more particularly preferred.

As outlined above, amino acid substitutions generally are based on the relative similarity of the amino acid side-chain substituents, for example, their hydrophobicity, hydrophilicity, charge, size, and the like. Exemplary substitutions that take into consideration the various foregoing characteristics are well known to those of skill in the art and include: arginine and lysine; glutamate and aspartate; serine and threonine; glutamine and asparagine; and valine, leucine and isoleucine.

One can also modify the internal amino acids, and/or amino and/or carboxy termini of polypeptide or peptide compounds of the invention to produce other compounds of the invention, i.e. polypeptide or peptide derivatives. Amino terminus modifications include methylation (e.g., —NHCH₃ or —N(CH₃)₂), acetylation (e.g., with acetic acid or a halogenated derivative thereof such as α-chloroacetic acid, α-bromoacetic acid, or α-iodoacetic acid), adding a benzyloxycarbonyl (Cbz) group, or blocking the amino terminus with any blocking group containing a carboxylate functionality defined by RCOO— or sulfonyl functionality defined by R—SO₂—, where R is selected from alkyl, aryl, heteroaryl, alkyl aryl, and the like, and similar groups. One can also incorporate a desamino acid at the N-terminus (so that there is no N-terminal amino group) to decrease susceptibility to proteases or to restrict the conformation of the polypeptide or peptide compound.

Carboxy terminus modifications include replacing the free acid with a carboxamide group or forming a cyclic lactam at the carboxy terminus to introduce structural constraints. One can also cyclize the peptides of the invention, or incorporate a desamino or descarboxy residue at the termini of the peptide, so that there is no terminal amino or carboxyl group, to decrease susceptibility to proteases or to restrict the conformation of the peptide. C-terminal functional groups of the compounds of the present invention include amide, amide lower alkyl, amide di(lower alkyl), lower alkoxy, hydroxy, and carboxy, and the lower ester derivatives thereof, and the pharmaceutically acceptable salts thereof.

One can replace the naturally occurring side chains of the 20 genetically encoded amino acids (or the stereoisomeric D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclic. In particular, proline analogues in which the ring size of the proline residue is changed from 5 members to 4, 6, or 7 members can be employed. Cyclic groups can be saturated or unsaturated, and if unsaturated, can be aromatic or non-aromatic. Heterocyclic groups preferably contain one or more nitrogen, oxygen, and/or sulfur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

One can also readily modify polypeptides or peptides by phosphorylation, and other methods (e.g., as described in Hruby, et al., 1990).

The peptide compounds of the invention also serve as structural models for non-peptidic compounds with similar biological activity. Those of skill in the art recognize that a variety of techniques are available for constructing compounds with the same or similar desired biological activity as the lead peptide compound, but with more favorable activity than the lead with respect to solubility, stability, and susceptibility to hydrolysis and proteolysis (See, Morgan and Gainor, 1989). These techniques include replacing the peptide backbone with a backbone composed of phosphonates, amidates, carbamates, sulfonamides, secondary amines, and N-methylamino acids.

Furthermore, the compounds of the present invention may contain one or more intramolecular disulfide bonds. In one embodiment, a peptide monomer or dimer comprises at least one intramolecular disulfide bond. In preferred embodiments, a peptide dimer comprises two intramolecular disulfide bonds. Such disulfide bonds may be formed by oxidation of the cysteine residues of the peptide core sequence. In one embodiment the control of cysteine bond formation is exercised by choosing an oxidizing agent of the type and concentration effective to optimize formation of the desired isomer. For example, oxidation of a peptide dimer to form two intramolecular disulfide bonds (one on each peptide chain) is preferentially achieved (over formation of intermolecular disulfide bonds) when the oxidizing agent is DMSO.

In certain embodiments, the formation of cysteine bonds is controlled by the selective use of thiol-protecting groups during peptide synthesis. For example, where a dimer with two intramolecular disulfide bonds is desired, the first monomer peptide chain is synthesized with the two cysteine residues of the core sequence protected with a first thiol protecting group (e.g., trityl(Trt), allyloxycarbonyl (Alloc), and 1-(4,4-dimethyl-2,6-dioxocyclohex-1-ylidene)ethyl (Dde) or the like), then the second monomer peptide is synthesized the two cysteine residues of the core sequence protected with a second thiol protecting group different from the first thiol protecting group (e.g., acetamidomethyl (Acm), t-butyl (tBu), or the like). Thereafter, the first thiol protecting groups are removed effecting bisulfide cyclization of the first monomer, and then the second thiol protecting groups are removed effecting bisulfide cyclization of the second monomer.

Other embodiments of this invention provide for analogs of these disulfide derivatives in which one of the sulfurs has been replaced by a CH₂ group or other isotere for sulfur. These analogs can be prepared from the compounds of the present invention, wherein each core sequence contains at least one Cys (C) or homocysteine residue and an α-amino-γ-butyric acid in place of the second C residue, via an intramolecular or intermolecular displacement, using methods known in the art (See, e.g., Barker et al., 1992 and Or et al., 1991). One of skill in the art will readily appreciate that this displacement can also occur using other homologs of α-amino-γ-butyric acid and homocysteine.

In addition to the foregoing cyclization strategies, other non-disulfide peptide cyclization strategies can be employed. Such alternative cyclization strategies include, for example, amide-cyclization strategies as well as those involving the formation of thio-ether bonds. Thus, the compounds of the present invention can exist in a cyclized form with either an intramolecular amide bond or an intramolecular thio-ether bond. For example, a peptide may be synthesized wherein one cysteine of the core sequence is replaced with lysine and the second cysteine is replaced with glutamic acid. Thereafter a cyclic monomer may be formed through an amide bond between the side chains of these two residues. Alternatively, a peptide may be synthesized wherein one cysteine of the core sequence is replaced with lysine. A cyclic monomer may then be formed through a thio-ether linkage between the side chains of the lysine residue and the second cysteine residue of the core sequence. As such, in addition to disulfide cyclization strategies, amide-cyclization strategies and thio-ether cyclization strategies can both be readily used to cyclize the compounds of the present invention. Alternatively, the amino-terminus of the peptide can be capped with an α-substituted acetic acid, wherein the α-substituent is a leaving group, such as an α-haloacetic acid, for example, α-chloroacetic acid, α-bromoacetic acid, or α-iodoacetic acid.

Included with the below description, the U.S. patent application Ser. No. 10/844,933 and International Patent Application No. PCT/US04/14887, filed May 12, 2004, are incorporated by reference herein in their entirety. Water-soluble polymers, such as polyethylene glycol (PEG), can be used for the covalent modification of polypeptides or peptides of therapeutic importance. Attachment of such polymers is thought to enhance biological activity, increase aqueous solubility, and enhance resistance to protease digestion. For example, covalent attachment of PEG to therapeutic polypeptides such as interleukins (Knauf et al., 1988; Tsutsumi et al., 1995), interferons (Kita et al., 1990), catalase (Abuchowski et al., 1977), superoxide dismutase (Beauchamp et al., 1983), and adenosine deaminase (Chen et al., 1981), has been reported to extend their half life in vivo, and/or reduce their immunogenicity and antigenicity.

The polypeptide or peptide compounds of the invention may further comprise one or more water soluble polymer moieties. Preferably, these polymers are covalently attached to the polypeptide or peptide compounds. The water soluble polymer may be, for example, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), poly(n-vinyl pyrrolidone)polyethylene glycol, propropylene glycol homopolymers, polypropylene oxide/ethylene oxide copolymers, and polyoxyethylated polyols.

Polypeptides and peptides and other peptide-based molecules of the invention can be attached to water-soluble polymers (e.g., PEG) using any of a variety of chemistries to link the water-soluble polymer(s) to the receptor-binding portion of the molecule (e.g., peptide+spacer). A typical embodiment employs a single attachment junction for covalent attachment of the water soluble polymer(s) to the receptor-binding portion, however in alternative embodiments multiple attachment junctions may be used, including further variations wherein different species of water-soluble polymer are attached to the receptor-binding portion at distinct attachment junctions, which may include covalent attachment junction(s) to the spacer and/or to one or both peptide chains.

PEG reagents include, but are not limited to mPEG2-NHS, mPEG2-ALD, multi-Arm PEG, mPEG(MAL)₂, mPEG2(MAL), mPEG-NH₂, mPEG-SPA, mPEG-SBA, mPEG-thioesters, mPEG-Double Esters, mPEG-BTC, mPEG-ButyrALD, mPEG-ACET, heterofunctional PEGs (NH₂-PEG-COOH, Boc-PEG-NHS, Fmoc-PEG-NHS, NHS-PEG-VS, NHS-PEG-MAL), PEG acrylates (ACRL-PEG-NHS), PEG-phospholipids (e.g., mPEG-DSPE), multiarmed PEGs of the SUNBRITE series including the GL series of glycerine-based PEGs activated by a chemistry chosen by those skilled in the art, any of the SUNBRITE activated PEGs (including but not limited to carboxyl-PEGs, p-NP-PEGs, Tresyl-PEGs, aldehyde PEGs, acetal-PEGs, amino-PEGs, thiol-PEGs, maleimido-PEGs, hydroxyl-PEG-amine, amino-PEG-COOH, hydroxyl-PEG-aldehyde, carboxylic anhydride type-PEG, functionalized PEG-phospholipid, and other similar and/or suitable reactive PEGs as selected by those skilled in the art for their particular application and usage.

The number of polymer molecules attached may vary; for example, one, two, three, or more polymers may be attached to a polypeptide or peptide of the invention. The multiple attached polymers may be the same or different chemical moieties (e.g., PEGs of different molecular weight). In some cases, the degree of polymer attachment (the number of polymer moieties attached to a peptide and/or the total number of peptides to which a polymer is attached) may be influenced by the proportion of polymer molecules versus peptide molecules in an attachment reaction, as well as by the total concentration of each in the reaction mixture. In general, the optimum polymer versus peptide ratio (in terms of reaction efficiency to provide for no excess unreacted peptides and/or polymer moieties) will be determined by factors such as the desired degree of polymer attachment (e.g., mono, di-, tri-, etc.), the molecular weight of the polymer selected, whether the polymer is branched or unbranched, and the reaction conditions for a particular attachment method.

In other aspects, a polypeptide or peptide of the invention can be derivatized by the addition of water insoluble polymers. Representative water-insoluble polymers include, but are not limited to, polyphosphazines, poly(vinyl alcohols), polyamides, polycarbonates, polyalkylenes, polyacrylamides, polyalkylene glycols, polyalkylene oxides, polyalkylene terephthalates, polyvinyl ethers, polyvinyl esters, polyvinyl halides, polyvinylpyrrolidone, polyglycolides, polysiloxanes, polyurethanes, poly(methyl methacrylate), poly(ethyl methacrylate), poly(butyl methacrylate), poly(isobutyl methacrylate), poly(hexyl methacrylate), poly(isodecyl methacrylate), poly(lauryl methacrylate), poly(phenyl methacrylate), poly(methyl acrylate), poly(isopropyl acrylate), poly(isobutyl acrylate), poly(octadecyl acrylate) polyethylene, polypropylene, poly(ethylene glycol), poly(ethylene oxide), poly (ethylene terephthalate), poly(vinyl acetate), polyvinyl chloride, polystyrene, polyvinyl pyrrolidone, pluronics and polyvinylphenol and copolymers thereof.

Synthetically modified natural polymers of use in derivatives of the invention include, but are not limited to, alkyl celluloses, hydroxyalkyl celluloses, cellulose ethers, cellulose esters, and nitrocelluloses. Members of the broad classes of synthetically modified natural polymers include, but are not limited to, methyl cellulose, ethyl cellulose, hydroxypropyl cellulose, hydroxypropyl methyl cellulose, hydroxybutyl methyl cellulose, cellulose acetate, cellulose propionate, cellulose acetate butyrate, cellulose acetate phthalate, carboxymethyl cellulose, cellulose triacetate, cellulose sulfate sodium salt, and polymers of acrylic and methacrylic esters and alginic acid.

In certain aspects a polypeptide or peptide of the invention can be modified or derivatized by addition of saccharide groups, or modified sugars. The present invention provides for polypeptide and peptide derivatives that contain modified sugars, modified sugar nucleotides and conjugates of the modified sugars. In modified sugar compounds of the invention, the sugar moiety is preferably a saccharide, a deoxy-saccharide, an amino-saccharide, or an N-acyl saccharide. The term “saccharide” and its equivalents, “saccharyl,” “sugar,” and “glycosyl” refer to monomers, dimers, oligomers and polymers. The sugar moiety can also be functionalized with a modifying group. The modifying group is conjugated to the sugar moiety, typically, through conjugation with an amine, sulfhydryl or hydroxyl, e.g., primary hydroxyl, moiety on the sugar. In one embodiment, the modifying group is attached through an amine moiety on the sugar, e.g., through an amide, a urethane or a urea that is formed through the reaction of the amine with a reactive derivative of the modifying group.

Any sugar can be utilized as the sugar for conjugates of the invention. Such sugars include, but are not limited to, glucose, galactose, mannose, fucose, and sialic acid. Other useful sugars include amino sugars such as glucosamine, galactosamine, mannosamine, the 5-amine analogue of sialic acid and the like. The sugar can be a structure found in nature or it can be modified to provide a site for conjugating an additional modifying group.

Those of skill in the art will recognize that the structures and compositions set forth are generally applicable across the genus of saccharide groups, modified saccharide groups, activated modified saccharide groups and conjugates of modified saccharide groups.

### III. STIMULATION OF LUNG DEFENSES

The inventors have used the mouse as model for microbial infection of the lung. In certain studies, untreated mice had mortality of 100%, but treated mice were highly protected. Not be held to any particular mechanism or theory, it is believed that protection is due to activation of local defenses. The effects of single and repetitive exposure of a subject to a composition of the invention have been determined and no obvious gross pathology, such as premature death, weight loss, or behavioral changes have been observed.

Preclinical studies have been conducted to define the efficacy, mechanism, and toxicity of a composition and related methods of the invention. One benefit of the present invention is that it can be delivered and have effect quickly and easily. Also, the compositions can be produced economically in large quantities and easily stored, as well as easily transported by a person outside of a hospital setting. Typically, the administration of the inventive compositions and the methods of the invention result in at least some killing or inhibition of the invading pathogens even before cellular entry. In the case that some pathogens do enter cells in the lungs either by escaping extracellular killing or because the compositions are administered after pathogen exposure (preemptively) instead of before pathogen exposure (preventatively), it is contemplated that the compositions and related methods promote intracellular killing resulting from the enhanced or augmented local responses in the lungs. The compositions and related methods are contemplated to have or produce protective or therapeutic responses against a variety of respiratory pathogens.

The protection or therapy afforded an individual by a StIR polypeptide or peptide, e.g., EF2505 polypeptide or peptide, may be extended to additional classes of microbial pathogens including gram negative bacteria, intracellular bacteria, fungi, and viruses because of the broad activity of the antimicrobial mechanisms of the respiratory tract. An agent such as that described in this application would simplify countermeasure stockpiling and deployment. Also, the compositions and methods of the invention would eliminate the difficulty of rapidly identifying a specific pathogen during a bioweapon attack or other exposure or potential exposure event. In addition, the economic advantages of producing and purchasing an agent with applicability in multiple civilian and biodefense settings are significant. Augmenting local epithelial mechanisms is particularly attractive in subjects who often have neutropenia or impaired adaptive immune function, e.g., immune compromised subjects. The methods typically act locally rather than systemically, and provide broad effects against multiple pathogens. The effects are rapid and are attractive in a biodefense and epidemic setting.

Augmentation of innate defense capabilities of the lungs in normal hosts would be valuable during influenza or emergent respiratory viral epidemics for which adaptive immune vaccines are not available. Bacterial outbreaks with emergent or drug-resistant organisms might also be a situation in which boosting innate lung defenses could be helpful. Similarly, protection of caregivers during an epidemic would facilitate care of the sick while limiting spread.

Many people in the community live with chronically compromised defenses against infection, such as patients with diabetes and patients taking immunosuppressive drugs for autoimmune diseases or to prevent transplant rejection. These people might particularly benefit from augmentation of lung defenses during epidemics. Even more strikingly, cancer patients undergoing chemotherapy who have transient but severe compromise of immune defenses might benefit from transient protection. Pneumonia is a common occurrence in these patients, and is the leading cause of infectious death. Many chemotherapy drugs, such as alkylating agents and nucleoside analogs, cause severe transient neutropenia. Initially, neutropenic patients are susceptible to bacterial pneumonia from organisms seen in normal hosts, as well as bacteria of low virulence such as Stenotrophomonas maltophilia. With prolonged neutropenia, patients also become susceptible to infection with fungi of low virulence, particularly Aspergillus species.

Defenses of the lung can be stimulated to provide transient protection during prolonged periods of neutropenia. Other cancer patients, such as those receiving fludarabine or anti-lymphocyte antibodies, or those receiving calcineurin inhibitors and steroids after hematopoietic stem cell transplantation, have impaired adaptive immunity. These patients might also benefit from episodic stimulation of lung immunity to protect against invasion by fungi and bacteria that have colonized the airways, or to protect against epidemic viruses. Community outbreaks of seasonal respiratory “cold” viruses such as parainfluenza and RSV can cause fatal pneumonia in these compromised patients, and infection with many of these viruses can be rapidly identified from nasal washings.

Immune responses are divided into two categories in vertebrate animals: innate and adaptive immunity. Upon infection, recognition of microorganisms is primarily mediated by a set of germline-encoded molecules on innate immune cells that are referred to as pattern recognition receptors (PRRs) (Medzhitov and Janeway, Jr., 1997). These pattern recognition receptors are expressed as either membrane-bound or soluble proteins that recognize invariant molecular structures, called pathogen-associated molecular patterns (PAMPs) (Janeway, Jr. and Medzhitov, 2002). Pathogen-associated molecular patterns are unique, conserved, and essential microbial components, such as LPS, that are structurally different from host molecules (Medzhitov and Janeway, Jr., 1997; Janeway, Jr. and Medzhitov, 2002).

Most multicellular organisms possess an “innate immune system” that does not change during the lifetime of the organism. In contrast, adaptive immunity is the responses to pathogens that change and develop during the lifetime of an individual. Organisms that possess an adaptive immunity also possess an innate immunity, and with many of the mechanisms between the systems being common, it is not always possible to draw a hard and fast boundary between the individual components involved in each, despite the clear difference in operation. Higher vertebrates and all mammals have both an innate and an adaptive immune system.

A. Innate Immune System.

The adaptive immune system may take days or weeks after an initial infection to have an effect. However, most organisms are under constant assault from pathogens that must be kept in check by the faster-acting innate immune system. Innate immunity defends against pathogens by rapid responses coordinated through “innate” mechanisms that recognize a wide spectrum of conserved pathogenic components. Plants and many lower animals do not possess an adaptive immune system, and rely instead on their innate immunity. Substances of both microbial and non-microbial sources are able to stimulate innate immune responses

The innate immune system, when activated, has a wide array of effector cells and mechanisms. There are several different types of phagocytic cells, which ingest and destroy invading pathogens. The most common phagocytes are neutrophils, macrophages, and dendritic cells. Another cell type, natural killer cells are especially adept at destroying cells infected with viruses. Another component of the innate immune system is known as the complement system. Complement proteins are normally inactive components of the blood. However, when activated by the recognition of a pathogen or antibody, the various proteins are activated to recruit inflammatory cells, coat pathogens to make them more easily phagocytosed, and to make destructive pores in the surfaces of pathogens.

The “first-line” defense includes physical and chemical barriers to infection, such as skin and mucus coating of the gut and airways, physically preventing the interaction between the host and the pathogen. Pathogens, which penetrate these barriers, encounter constitutively-expressed anti-microbial molecules (e.g., lysozyme) that restrict the infection. The “second-line” defense includes phagocytic cells (macrophages and neutrophil granulocytes) that can engulf (phagocytose) foreign substances.

Phagocytosis involves chemotaxis, where phagocytic cells are attracted to microorganisms by means of chemotactic chemicals such as microbial products, complement, damaged cells and white blood cell fragments. Chemotaxis is followed by adhesion, where the phagocyte sticks to the microorganism. Adhesion is enhanced by opsonization, where proteins like opsonins are coated on the surface of the bacterium. This is followed by ingestion, in which the phagocyte extends projections, forming pseudopods that engulf the foreign organism. Finally, the pathogen is digested by the enzymes in the lysosome, involving reactive oxygen species and proteases.

In addition, anti-microbial proteins may be activated if a pathogen passes through a physical barrier. There are several classes of antimicrobial proteins, such as acute phase proteins (e.g., C-reactive protein, which enhances phagocytosis and activates complement when it binds the C-protein of S. pneumoniae), lysozyme, and the complement system).

The complement system is a very complex group of serum proteins, which is activated in a cascade fashion. Three different pathways are involved in complement activation: (a) a classical pathway that recognizes antigen-antibody complexes, (b) an alternative pathway that spontaneously activates on contact with pathogenic cell surfaces, and (c) a mannose-binding lectin pathway that recognizes mannose sugars, which tend to appear only on pathogenic cell surfaces. A cascade of protein activity follows complement activation; this cascade can result in a variety of effects, including opsonization of the pathogen, destruction of the pathogen by the formation and activation of the membrane attack complex, and inflammation.

Interferons are also anti-microbial proteins. These molecules are proteins that are secreted by virus-infected cells. These proteins then diffuse rapidly to neighboring cells, inducing the cells to inhibit the spread of the viral infection. Essentially, these anti-microbial proteins act to prevent the cell-to-cell proliferation of viruses.

B. Adaptive Immune System

The adaptive immune system, also called the “acquired immune system,” ensures that most mammals that survive an initial infection by a pathogen are generally immune to further illness, caused by that same pathogen. The adaptive immune system is based on dedicated immune cells termed leukocytes (white blood cells) that are produced by stem cells in the bone marrow, and mature in the thymus and/or lymph nodes. In many species, including mammals, the adaptive immune system can be divided into: (a) a humoral immune system that acts against bacteria and viruses in the body liquids (e.g., blood) by means of proteins, called immunoglobulins (also known as antibodies), which are produced by B cells; and (b) a cellular immune system that destroys virus-infected cells (among other duties) with T cells (also called “T lymphocytes”; “T” means they develop in the thymus). The adaptive immune system is typically directed toward a specific pathogen, e.g., vaccination.

### IV. MICROBIAL ORGANISMS

Embodiments of the invention include compositions and related methods for a broad protection against a variety of pathogens or potential pathogens. For example, bacterial pneumonia in a normal host occurs at a rate of 1/100 persons/year, mostly in elderly adults and young children and can be caused by a variety of organisms. It is most commonly caused by Streptococcus pneumoniae, followed in frequency by encapsulated Hemophilus influenzae. Other bacteria such as enteric gram negatives, anaerobes, and Staphylococcus aureus are significant causes of pneumonia in specific settings, such as healthcare facilities. Mycobacterium tuberculosis is highly infectious, and historically was an important cause of mortality worldwide. It has mostly been controlled with antibiotics in the developed world, though multidrug-resistant strains continue to cause problems and are classified as Category C bioweapon agents. Legionella pneumophila was first identified during an outbreak in Philadelphia in 1978, though it is now recognized to occur widely at a low endemic rate related to environmental sources. Also, fungal infections of the lungs can cause symptomatic disease in normal hosts. Histoplasma capsulatum, Coccidiodes immitis, Blastomyces dermatitidis, and Cryptococcus neoformans can all cause pneumonia related to local exposure to high environmental concentrations. Pneumonia due to these pathogenic fungi is usually self-limited in normal hosts. Some additional “atypical” microorganisms, such as mycoplasmas, account for a substantial fraction of additional pneumonias in normal hosts. It is contemplated that a composition of the present invention can provide a rapid, temporal protection against a spectrum of agents that can cause, for example pneumonia or other disease states. In certain aspects the present invention may be used in combination with a vaccination regime to provide an additional protection to a subject that may or is exposed to one or more pathogenic or potentially pathogenic organism.

In particular aspects of the invention the compositions and methods of the invention may be used to prevent, reduce the risk of or the treat infection or exposure to a biological weapon or intentional exposure of a subject(s) to an inhaled infective agent. The only microbial pathogen that has been used as a terrorist weapon in the modern era is Bacillus anthracis, which has a case-fatality rate of 75% when infection occurs by the respiratory route, even with the use of appropriate antibiotics. Francisella tularensis is an aerobic, gram negative coccobacillus that is a facultative intracellular pathogen. It is highly infectious, highly pathogenic, and survives under harsh environmental conditions, making it a serious bioterror threat even though it is poorly transmissible from person to person (Dennis, 2001). A vaccine is available, but is only partially protective. The World Health Organization estimated that aerosol dispersal of 50 kg of virulent Francisella tularensis over a metropolitan area with 5 million inhabitants would result in 250,000 incapacitating casualties, including 19,000 deaths; the Centers for Disease Control (CDC) estimated the economic cost of such an attack to be $5.4 billion for every 100,000 persons exposed (Dennis, 2001).

Other Class A bioterrorism agents that can be transmitted by aerosol are Yersinia pestis, smallpox virus, and hemorrhagic fever viruses. In addition, multiple Class B and C agents can be effectively delivered by the respiratory route. Together, these organisms comprise gram-positive, gram-negative, intracellular, and extracellular bacteria, as well as a variety of viral classes. Because of the potential difficulty in initially identifying a specific bioterrorism agent, the complexity of locally stockpiling adaptive immune vaccines and antibiotics directed at specific agents, and the remarkable virulence of organisms such as Bacillus anthracis despite appropriate treatment, stimulation of innate defense capabilities of the lungs that could either prevent or preempt infection with a bioterror agent delivered by the respiratory route could have great public health value.

A. Pathogenic or Potentially Pathogenic Microbes

There are numerous microbes that are considered pathogenic or potentially pathogenic under certain conditions. In certain aspects, the pathogenicity is determined relative to infection via the lungs. Bacterial microbes include, but are not limited to various species of the Bacillus, Yersinia, Franscisella, Streptococcus, Staphylococcus, Pseudomonas, Mycobacterium, Burkholderia genus of bacteria. Particular species of bacteria from which a subject may be protected include, but is not limited to Bacillus anthracia, Yersinia pestis, Francisella tularensis, Streptococcus pnemoniae, Staphylococcus aureas, Pseudomonas aeruginosa, Burkholderia cepacia, Corynebacterium diphtheriae, Clostridia spp, Shigella spp., Mycobacterium avium, M. intracellulare, M. kansasii, M. paratuberculosis, M. scrofulaceum, M. simiae, M. habana, M. interjectum, M. xenopi, M. heckeshornense, M. szulgai, M. fortuitum, M. immunogenum, M. chelonae, M. marinum, M. genavense, M. haemophilum, M. celatum, M. conspicuum, M. malmoense, M. ulcerans, M. smegmatis, M. wolinskyi, M. goodii, M. thermoresistible, M. neoaurum, M. vaccae, M. palustre, M. elephantis, M. bohemicam and M. septicum.

B. Viruses

There are numerous viruses and viral strains that are considered pathogenic or potentially pathogenic under certain conditions. Viruses can be placed in one of the seven following groups: Group I: double-stranded DNA viruses, Group II: single-stranded DNA viruses, Group III: double-stranded RNA viruses, Group IV: positive-sense single-stranded RNA viruses, Group V: negative-sense single-stranded RNA viruses, Group VI: reverse transcribing Diploid single-stranded RNA viruses, Group VII: reverse transcribing Circular double-stranded DNA viruses. Viruses include the family Adenoviridae, Arenaviridae, Caliciviridae, Coronaviridae, Filoviridae, Flaviviridae, Hepadnaviridae, Herpesviridae (Alphaherpesvirinae, Betaherpesvirinae, Gammaherpesvirinae), Nidovirales, Papillomaviridae, Paramyxoviridae (Paramyxovirinae, Pneumovirinae), Parvoviridae (Parvovirinae, Picornaviridae), Poxyiridae (Chordopoxyirinae), Reoviridae, Retroviridae (Orthoretrovirinae), and/or Togaviridae. These virus include, but are not limited to various strains of influenza, such as avian flu (e.g., H5N1). Particular virus from which a subject may be protected include, but is not limited to Cytomegalovirus, Respiratory syncytial virus and the like.

Examples of pathogenic virus include, but are not limited to Influenza A, H5N1, Marburg, Ebola, Dengue, Severe acute respiratory syndrome coronavirus, Yellow fever virus, Human respiratory syncytial virus, Vaccinia virus and the like.

C. Fungi

There are numerous fungal species that are considered pathogenic or potentially pathogenic under certain conditions. Protection can be provided for, but not limited to Aspergillus fumigatus, Candida albicans, Cryptococcus neoformans, Histoplasma capsulatum, Coccidioides immitis, or Pneumocystis carinii, and/or Blastomyces dermatitidis.

### V. FORMULATIONS AND ADMINISTRATION

The pharmaceutical compositions disclosed herein may be administered via the respiratory system of a subject. StIR polypeptides or peptides may be prepared in water suitably mixed with a surfactant, such as hydroxypropylcellulose. Dispersions may also be prepared in glycerol, liquid polyethylene glycols and mixtures thereof, and in oils. Under ordinary conditions of storage and use, these preparations contain a preservative to prevent the growth of microorganisms. The pharmaceutical forms suitable for inhalation include sterile aqueous solutions or dispersions and sterile powders for the extemporaneous preparation of sterile inhalable solutions or dispersions. In all cases the form is typically sterile and capable of inhalation directly or through some intermediary process or device. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. The prevention of the action of microorganisms can be brought about by various antibacterial and antifungal agents, for example, parabens, chlorobutanol, phenol, sorbic acid, thimerosal, and the like.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards or other similar organizations.

Sterile compositions are prepared by incorporating the active components in the required amount in the appropriate solvent with various other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile compositions, some methods of preparation are vacuum-drying and freeze-drying techniques which yield a powder of the active ingredient plus any additional desired ingredient from a previously sterile-filtered solution thereof.

Pulmonary/respiratory drug delivery can be implemented by different approaches, including liquid nebulizers, aerosol-based metered dose inhalers (MDI's), sprayers, dry powder dispersion devices and the like. Such methods and compositions are well known to those of skill in the art, as indicated by U.S. Pat. Nos. 6,797,258, 6,794,357, 6,737,045, and 6,488,953, all of which are incorporated by reference. According to the invention, at least one pharmaceutical composition can be delivered by any of a variety of inhalation or nasal devices known in the art for administration of a therapeutic agent by inhalation. Other devices suitable for directing pulmonary or nasal administration are also known in the art. Typically, for pulmonary administration, at least one pharmaceutical composition is delivered in a particle size effective for reaching the lower airways of the lung or sinuses.

All such inhalation devices can be used for the administration of a pharmaceutical composition in an aerosol. Such aerosols may comprise either solutions (both aqueous and non aqueous) or solid particles. Metered dose inhalers typically use a propellant gas and require actuation during inspiration. See, e.g., WO 98/35888; WO 94/16970. Dry powder inhalers use breath-actuation of a mixed powder. See U.S. Pat. Nos. 5,458,135; 4,668,218; PCT publications WO 97/25086; WO 94/08552; WO 94/06498; and European application EP 0237507, each of which is incorporated herein by reference in their entirety. Nebulizers produce aerosols from solutions, while metered dose inhalers, dry powder inhalers, and the like generate small particle aerosols. Suitable formulations for administration include, but are not limited to nasal spray or nasal drops, and may include aqueous or oily solutions of a StIR polypeptide or peptide.

A spray comprising a pharmaceutical composition of the present invention can be produced by forcing a suspension or solution of a composition through a nozzle under pressure. The nozzle size and configuration, the applied pressure, and the liquid feed rate can be chosen to achieve the desired output and particle size. An electrospray can be produced, for example, by an electric field in connection with a capillary or nozzle feed.

A pharmaceutical composition of the present invention can be administered by a nebulizer such as a jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the composition creating an aerosol.

In a metered dose inhaler (MDI), a propellant, a composition, and any excipients or other additives are contained in a canister as a mixture with a compressed gas. Actuation of the metering valve releases the mixture as an aerosol.

Pharmaceutical compositions for use with a metered-dose inhaler device will generally include a finely divided powder containing a composition of the invention as a suspension in a non-aqueous medium, for example, suspended in a propellant with the aid of a surfactant. The propellant can be any conventional material employed for this purpose such as chlorofluorocarbon, a hydrochlorofluorocarbon, a hydrofluorocarbon, or a hydrocarbon including trichlorofluoromethane, dichlorodifluoromethane, dichlorotetrafluoroethanol and 1,1,1,2-tetrafluoroethane, HFA-134a (hydrofluoroalkane-134a), HFA-227 (hydrofluoroalkane-227), or the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically acceptable” refers to molecular entities and compositions that do not produce an allergic or similar untoward reaction when administered to a subject. The preparation of an aqueous composition that contains a polypeptide or peptide as an active ingredient is well understood in the art.

### VI. COMBINATION TREATMENTS

The compositions and methods of the present invention may be used in the context of a number of therapeutic or prophylactic applications. In order to increase the effectiveness of a treatment with the compositions of the present invention, e.g., StIR polypeptides or peptides, or to augment the protection of another therapy (second therapy), e.g., vaccination or antimicrobial therapy, it may be desirable to combine these compositions and methods with other agents and methods effective in the treatment, reduction of risk of infection, or prevention of diseases and pathologic conditions, for example, anti-bacterial, anti-viral, and/or anti-fungal treatments.

Various combinations may be employed; for example, a StIR polypeptide or peptide or variant or derivative or analog thereof, such as EF2505 polypeptide or peptide or variant or derivative or analog thereof, is “A” and the secondary therapy is “B”:

A/B/A B/A/B B/B/A A/A/B A/B/B B/A/A A/B/B/B B/A/B/B
B/B/B/A B/B/A/B A/A/B/B A/B/A/B A/B/B/A B/B/A/A
B/A/B/A B/A/A/B A/A/A/B B/A/A/A A/B/A/A A/A/B/A

Administration of a composition of the present invention to a subject will follow general protocols for the administration via the respiratory system, and the general protocols for the administration of a particular secondary therapy will also be followed, taking into account the toxicity, if any, of the treatment. It is expected that the treatment cycles would be repeated as necessary. It also is contemplated that various standard therapies, as well as vaccination, may be applied in combination with the described therapies.

A. Anti-Virals

In certain aspects of the invention an anti-viral agent may be used in combination with a StIR polypeptide or peptide. Antiviral drugs are a class of medication used specifically for treating viral infections. Like antibiotics for bacteria, specific antivirals are used for specific viruses. They should be distinguished from viricides, which actively deactivate virus particles outside the body. Most of the antivirals now available are designed to help deal with HIV, herpes viruses, the hepatitis B and C viruses, and influenza A and B viruses. Anti-viral agents useful in the invention include but are not limited to immunoglobulins, amantadine, interferons, nucleotide analogues, and protease inhibitors.

One anti-viral strategy is to interfere with the ability of a virus to infiltrate a target cell. This stage of viral replication can be inhibited by using agents which mimic the virus-associated protein (VAP) and bind to the cellular receptors. Or by using agents which mimic the cellular receptor and bind to the VAP. This includes anti-VAP antibodies, receptor anti-idiotypic antibodies, extraneous receptor and synthetic receptor mimics. Two such “entry-blockers,” amantadine and rimantadine, have been introduced to combat influenza.

A second approach to anti-viral therapy is to target the processes that synthesize virus components after a virus invades a cell. One way of doing this is to develop nucleotide or nucleoside analogues that look like the building blocks of RNA or DNA, but deactivate the enzymes that synthesize the RNA or DNA once the analog is incorporated. Nucleotide analogs include, but are not limited to ribivirin, vidarabine, acyclovir, gangcyclovir, zidovudine, didanosine, zalcitabine, stavudine, and lamivudine.

Yet another antiviral technique is a set of drugs based on ribozymes, which are enzymes that will cut apart viral RNA or DNA at selected sites. In their natural course, ribozymes are used as part of the viral manufacturing sequence, but these synthetic ribozymes are designed to cut RNA and DNA at sites that will disable them.

Some viruses include an enzyme known as a protease that cuts viral protein chains apart so they can be assembled into their final configuration. HIV includes a protease, and so considerable research has been performed to find “protease inhibitors” to attack HIV at that phase of its life cycle. Protease inhibitors became available in the 1990s and have proven effective, though they can have unusual side effects, for example causing fat to build up in unusual places. Improved protease inhibitors are now in development.

The final stage in the life cycle of a virus is the release of completed viruses from the host cell, and this step has also been targeted by antiviral drug developers. Two drugs named zanamivir (RELENZA™) and oseltamivir (TAMIFLU™) that have been introduced to treat influenza prevent the release of viral particles by blocking a molecule named neuraminidase that is found on the surface of flu viruses, and also seems to be constant across a wide range of flu strains.

Anti-viral agents include, but are not limited to Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In certain embodiments an anti-viral is ribivirin and high dose ribivirin. Ribavirin is an anti-viral drug that is active against a number of DNA and RNA viruses. It is a member of the nucleoside antimetabolite drugs that interfere with duplication of viral genetic material. Though not effective against all viruses, ribavirin has wide range of activity, including important activities against influenzas, flaviviruses, and agents of many viral hemorrhagic fevers.

Typically, the oral form of ribavirin is used in the treatment of hepatitis C, in combination with pegylated interferon drugs. The aerosol form has been used in the past to treat respiratory syncytial virus-related diseases in children. However, its efficacy has been called into question by multiple studies, and most institutions no longer use it.

B. Anti-Bacterials

Examples of anti-bacterials include, but are not limited to, β-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other β-lactams (such as imipenem, monobactams), β-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines. Anti-bacterials also include, but are not limited to: Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexii Hydrochloride; Cephaloglycini; Cephaloridine; Cephalothin Sodium; Cephapirin Sodium; Cephradine; Cetocycline Hydrochloride; Cetophenicol; Chloramphenicol; Cliloramphenicol Palmitate; Chloramphenicol Pantotheniate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Clarithromycin; Clinafloxacin Hydrochloride; Clildamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycine; Demeclocycine Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Mezlocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifuirpirinol; Nifurquinazol; Nifurthiazole; Nitrocycline; Nitrofurantoin; Nitromide; Norfloxacin; Novobiocin. Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; and/or Zorbamycin.

C. Anti-Fungals

Anti-fungal agents include, but are not limited to, Myambutol (Ethambutol Hydrochloride), Dapsone (4,4′-diaminodiphenylsulfone), Paser Granules (aminosalicylic acid granules), rifapentine, Pyrazinamide, Isoniazid, Rifampin, Rifadin IV, Rifampin and Isoniazid, Rifampin, Isoniazid, and Pyrazinamide, Streptomycin Sulfate and Trecator-SC (Ethionamide).

D. Other Agents

In certain aspects of the invention an anti-inflammatory agent may be used in combination with StIR polypeptide or peptide or variant or derivative or analog.

Steroidal anti-inflammatories for use herein include, but are not limited to fluticasone, beclomethasone, any pharmaceutically acceptable derivative thereof, and any combination thereof. As used herein, a pharmaceutically acceptable derivative includes any salt, ester, enol ether, enol ester, acid, base, solvate or hydrate thereof. Such derivatives may be prepared by those of skill in the art using known methods for such derivatization.

Fluticasone—Fluticasone propionate is a synthetic corticosteroid and has the empirical formula C₂₅H₃₁F₃O₅S. It has the chemical name S-(fluoromethyl)6α,9-difluoro-11β-17-dihydroxy-16α-methyl-3-oxoandrosta-1,4-diene-17β-carbothioate,17-propionate. Fluticasone propionate is a white to off-white powder with a molecular weight of 500.6 and is practically insoluble in water, freely soluble in dimethyl sulfoxide and dimethylformamide, and slightly soluble in methanol and 95% ethanol.

In an embodiment, the formulations of the present invention may comprise a steroidal anti-inflammatory (e.g., fluticasone propionate)

Beclomethasone—In certain aspects the steroidal anti-inflammatory can be beclomethasone dipropionate or its monohydrate. Beclomethasone dipropionate has the chemical name 9-chloro-11b,17,21-trihydroxy-16b-methylpregna-1,4-diene-3,20-dione-17,21-dipropionate. The compound may be a white powder with a molecular weight of 521.25; and is very slightly soluble in water (Physicians' Desk Reference), very soluble in chloroform, and freely soluble in acetone and in alcohol.

Providing steroidal anti-inflammatories according to the present invention may enhance the compositions and methods of the invention by, for example, attenuating any unwanted inflammation. Examples of other steroidal anti-inflammatories for use herein include, but are not limited to, betamethasone, triamcinolone, dexamethasone, prednisone, mometasone, flunisolide and budesonide.

In accordance with yet another aspect of the invention, the non-steroidal anti-inflammatory agent may include aspirin, sodium salicylate, acetaminophen, phenacetin, ibuprofen, ketoprofen, indomethacin, flurbiprofen, diclofenac, naproxen, piroxicam, tebufelone, etodolac, nabumetone, tenidap, alcofenac, antipyrine, amimopyrine, dipyrone, animopyrone, phenylbutazone, clofezone, oxyphenbutazone, prexazone, apazone, benzydamine, bucolome, cinchopen, clonixin, ditrazol, epirizole, fenoprofen, floctafeninl, flufenamic acid, glaphenine, indoprofen, meclofenamic acid, mefenamic acid, niflumic acid, salidifamides, sulindac, suprofen, tolmetin, nabumetone, tiaramide, proquazone, bufexamac, flumizole, tinoridine, timegadine, dapsone, diflunisal, benorylate, fosfosal, fenclofenac, etodolac, fentiazac, tilomisole, carprofen, fenbufen, oxaprozin, tiaprofenic acid, pirprofen, feprazone, piroxicam, sudoxicam, isoxicam, celecoxib, Vioxx® and tenoxicam.

### VII. KITS

Any of the compositions described herein may be comprised in a kit. In a non-limiting example, reagents for delivery of a StIR peptide are included in a kit. In certain aspects the kit is portable and may be carried on a person much like an asthma inhaler is carried. The kit may further include a pathogen detector. The kit may also contain a gas or mechanical propellant for compositions of the invention.

The components of the kits may be packaged either in an aqueous, powdered, or lyophilized form. The container means of the kits will generally include at least one inhaler, canister, vial, test tube, flask, bottle, syringe or other container means, into which a component may be placed, and preferably, suitably aliquoted. Where there is more than one component in the kit (second agent, etc.), the kit also will generally contain a second, third or other additional container into which the additional components may be separately placed. However, various combinations of components may be comprised in a vial, canister, or inhaler. A container of the invention can include a canister or inhaler that can be worn on a belt or easily carried in a pocket, backpack or other storage container. The kits of the present invention also will typically include a means for containing the described compositions or their variations, and any other reagent containers in close confinement for commercial sale. Such containers may include injection or blow molded plastic containers into which the desired vials are retained.

When the components of the kit are provided in one and/or more liquid solutions, e.g., the liquid solution is an aqueous solution, with a sterile aqueous solution being particularly preferred, but not required. However, the components of the kit may be provided as dried powder(s). When reagents and/or components are provided as a dry powder, the powder may be reconstituted by the addition of a suitable solvent or administered in a powdered form. It is envisioned that a solvent may also be provided in another container means.

A kit will also include instructions for employing the kit components as well the use of any other reagent not included in the kit. Instructions may include variations that can be implemented.

It is contemplated that such reagents are embodiments of kits of the invention. Such kits, however, are not limited to the particular items identified above and may include any reagent used directly or indirectly in the detection of pathogenic microorganisms or administration of a StIR polypeptide or peptide of the invention.

### VIII. EXAMPLES

The following examples are given for the purpose of illustrating various embodiments of the invention and are not meant to limit the present invention in any fashion. One skilled in the art will appreciate readily that the present invention is well adapted to carry out the objects and obtain the ends and advantages mentioned, as well as those objects, ends and advantages inherent herein. The present examples, along with the methods described herein are presently representative of certain embodiments and are not intended as limitations on the scope of the invention. Changes therein and other uses which are encompassed within the spirit of the invention as defined by the scope of the claims will occur to those skilled in the art.

### Example 1

### Identification of an Enterococcal Protein Recognizing Repeated LRRs

Common gram-positive bacterial pathogens contain 15-50 cell wall anchored proteins, of which 30-50% contain segments composed of repeated IgG-like modules. These proteins appear to mediate adherences of the pathogen to host tissues and/or to counter host defense systems. Many of the gram-positive pathogens live primarily in the extra cellular space in the host and are therefore exposed to a sub-set of host toll-like receptors (TLRs). It is reasonable to assume that these bacteria have acquired the ability to modulate the innate immune system in order to survive in this environment.

There are proteins that interact with proteins composed of repeated leucine rich repeats, a structural motif that dominates the extracellular domains of the Toll like receptors and also occurs in NLRs. A screen of cell wall anchored proteins from E. faecalis identified the protein, EF2505, that bound multiple proteins composed of repeated leucine rich repeats. The leucine rich repeat binding site within EF2505 was located within a segment composed of IgG-like module number 2-3 that can be readily produced recombinantly and purified rapidly.

### Example 2

### EF2505 can Induce an NF-kB Response in Cultured Mammalian Cells and Protect Against Pulmonary Pathogens In Vivo

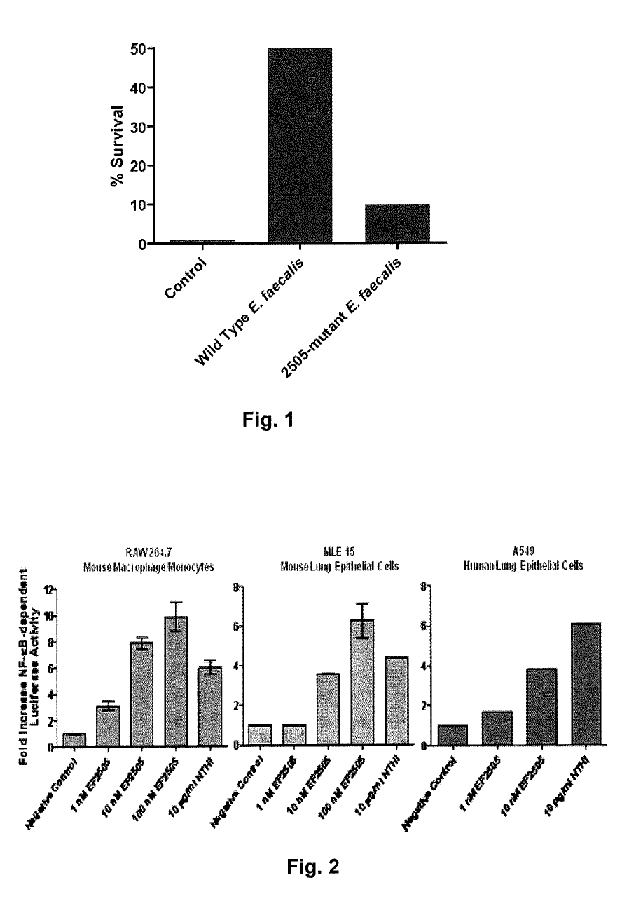

The protective effect is also seen with lysate from other pathogenic bacteria, including E. faecalis. Since it's likely EF2505 interacts with leucine rich repeat-containing PRRs, such as TLRs, the protective effect of bacterial lysates made from wild type E. faecalis bacteria or an isogenic EF2505 mutant was examined. The results of these experiments showed a dramatic difference in protective effect. The lysate from wild-type bacteria protected against S. pneumoniae challenge, however (FIG. 1) the lysate from the EF2505 KO did not.

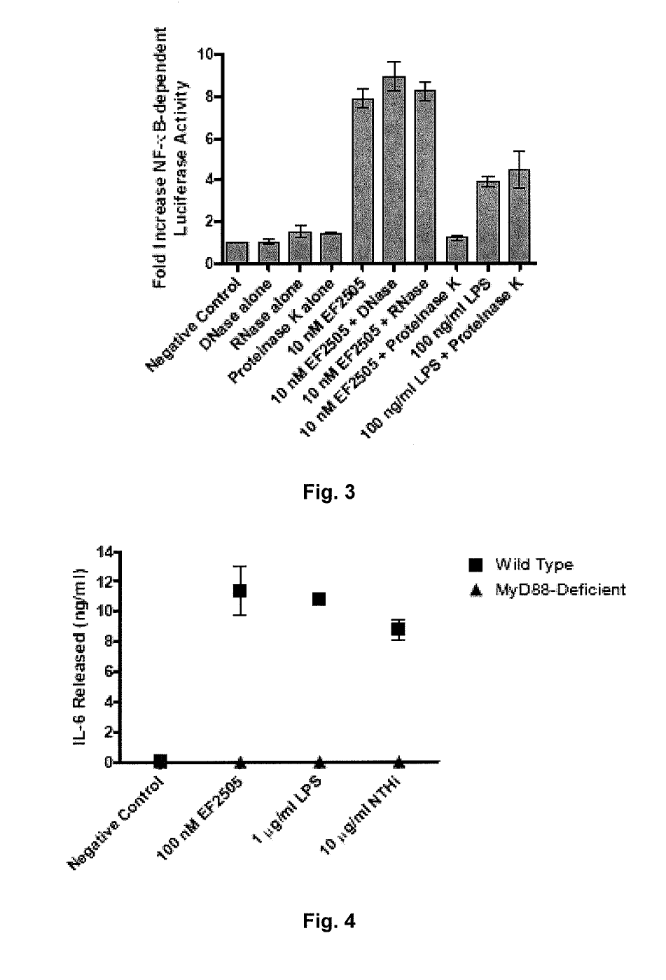

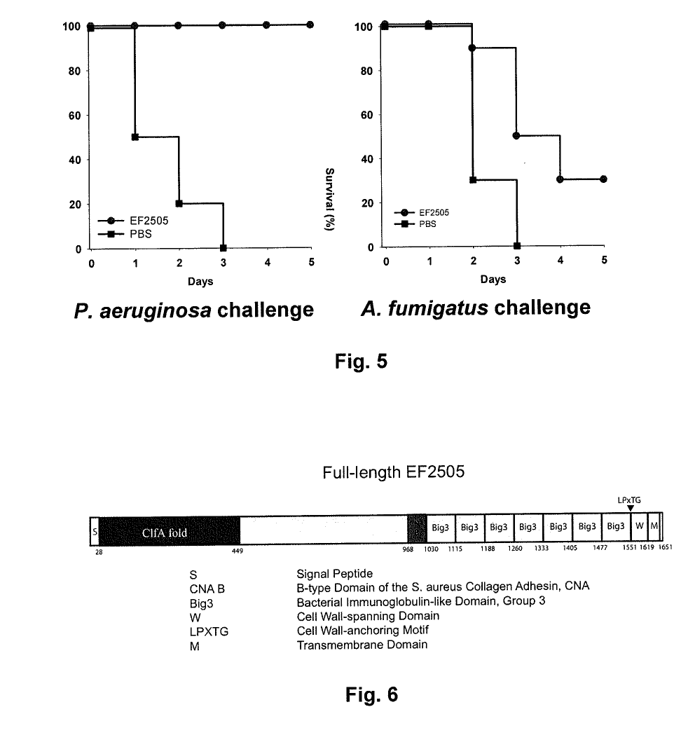

Recombinant EF2505 induced NF-kB activation in mammalian cells (FIG. 2). This activation was dose-dependent (FIG. 2). NF-kB activation appears to be due to purified EF2505 itself and not other potential contaminants such as LPS (FIG. 3). EF2505-stimulation also leads to increased pro-inflammatory cytokine release, an observation that supports the ability of EF2505 to activate NF-kB-dependent signaling (FIG. 4). This release of pro-inflammatory cytokines is dependent on the adaptor protein MyD88 (FIG. 4), suggesting that a TLR is involved.

The ability of EF2505 to protect against Pseudomonas aeruginosa-induced death was examined using the murine model described above. Mice were nebulized with 11 mg of purified EF2505 in PBS or PBS alone 24 hours prior to P. aeruginosa-exposure. As shown in FIG. 5, EF2505 pre-treatment significantly protected mice from P. aeruginosa-induced death compared with the PBS controls. Importantly, these data suggest that a single protein, EF2505, appears to have significant in vivo effects.

### Example 3

### Identify the Mammalian Cellular Receptor(s) that is/are Targeted by EF2505 to Induce NF-kB Activation

The present invention demonstrated that a single protein produced by Enterococcus faecalis, EF2505, is capable of inducing protection against P. aeruginosa infections in lungs (FIG. 5). The fact that the NF-kB signaling event caused by both EF2505 depends on the adaptor protein MyD88 (FIG. 4) indicates that a TLR, the IL-1 receptor, and/or the IL-18 receptor are responsible for recognizing EF2505.

To identify which receptor is targeted by this bacterial protein, macrophages from mice that are deficient in individual TLRs, the IL-1 receptor, or the IL-18 receptor with EF2505 are stimulated. Mice with knock-out mutations in TLR-2, TLR-3, TLR-4, IL-1 receptor, and the IL-18 receptor are available from the Jackson laboratories. Since TLR-2 forms a heterodimer with either TLR-1 or TLR-6, removing TLR-2 will also eliminate signaling through TLR-1 and TLR-6. TLR-5, -7, -8, -9, and -12 mice are available in the scientific community. TLR-10 is not expressed in mice; since this bacterial protein stimulates NF-kB signaling in murine cells, it is safe to say that this TLR is not the responsible receptor. Bone marrow macrophages from wild type or receptor-KO mice are isolated from bone marrow cells that are cultured in DMEM medium supplemented with 20% fetal bovine serum (FBS) and 30% supernatant derived from L929 confluent cells. At day 5 or 6, immature macrophages are collected and cultured in RPMI 1640 medium, 5% FBS. Macrophages (1×10⁶ cells ml⁻¹) are then cultured with media alone (negative control), various concentrations of EF2505, or 1 μg/ml LPS (positive control) for 24 h. Concentrations of IL-6 and TNFα (pro-inflammatory cytokines that are known to be produced in response to NF-kB activation; antibodies from PharMingen) in the cultured supernatants are measured by enzyme-linked immunosorbent assay (ELISA).

### Example 4

### NF-kB Signaling in Cells Transfected with Specific Receptors (Gain of Function)

To verify that the receptor identified in the experiments described is necessary for EF2505-dependent NF-kB activation, one may perform knock-in experiments with the identified receptor. Stable CHO-K1 cell lines expressing both the identified receptor and an NF-kB-dependent reporter construct are generated for this purpose. The CHO-K1 cells (2×10⁶) will be plated into 10-cm dishes and transfected the following day with 5 μg of PBIIX (NF-kB luciferase) DNA. All of the transfections are performed using Lipofectamine 2000 according to the manufacturer's instructions (Invitrogen). After 4 h, the medium is removed and the cells are supplemented with fresh medium containing 10% FBS. The next day, the cells are trypsinized, seeded at a lower density in 15-cm plates, and cultured in a selection medium containing 400 μg/ml G418. The medium is replaced twice a week for 3-4 weeks. G418-resistant clones are transferred individually to small plates and propagated in G418 medium before analysis. Similarly, the G418-resistant clones are re-transfected with the blasticidin-resistant vector pEF6 (Invitrogen) that encodes the identified receptor (cloned from RAW 264.7 cells). Double stable transfectants will be selected for in the presence of 400 μg/ml G418 and 2 μg/ml blasticidin. The clones are analyzed by immunoblotting to identify those that are expressing both the receptor and luciferase. We have successfully used this strategy to generate TLR11/NFkB-LUC/CHO and TLR13/NFkBLUC/CHO stable cell lines; one of these cells lines is used as a negative control for the experiment.

### Example 5

### Identifying EF2505 Associated Proteins Through Co-Immuno Precipitation

Immunoprecipitation assays are performed with wild type bone marrow-derived macrophages because they are stimulated by purified EF2505 and their use enables use of macrophages generated from receptor knock-out mice as a negative control for these experiments. Macrophages are incubated with purified EF2505 for 30 min at 37° C. Then, the conditioned media are removed, the cell layer are washed with PBS, and the macrophages are lysed in the presence of a detergent containing protease inhibitors. Half of the lysates are probed with pre-immune serum and the other half are probed with a rabbit anti-EF2505 antibody previously generated and characterized or an antibody that recognizes the receptor (Abcam) overnight at 4° C. The sample is incubated with protein A-agarose for 1 h at 4° C. with agitation. Following this incubation, the mixture is subjected to a brief centrifugation step. The pelleted protein A-agarose beads is washed extensively with cell lysis buffer containing protease inhibitors. The proteins bound to the protein A-agarose beads are eluted using SDS/PAGE reducing sample buffer. The sample is then run on SDS/PAGE for Western blot analysis to determine whether the anti-EF2505 antibody specifically pulls down the identified receptor and if the anti-receptor antibody specifically pulls down EF2505.

### Example 6

### Determine the Host Cell Signaling Pathways and Surface Receptors Required for Stimulated Innate Resistance by EF2505

The remarkable inducibility of lung mucosal innate immunity by treatment with isolated EF2505 was demonstrated. This phenomenon, termed Stimulated Innate Resistance (StIR), results in extremely broad protection against bacterial, fungal, and viral respiratory pathogens. The breadth of protection conferred by StIR suggests a multifaceted process, arising from multiple concurrently stimulated microbial-sensing pathways. To investigate such a phenomenon, whole genome microarray expression analysis was employed.

To verify that the molecules identified in the RNAi screen actually play a critical role in the ability of bacterial products to cause NF-kB activation, the ability of EF2505 to cause NF-kB-dependent pro-inflammatory cytokine release from murine macrophages that are deficient in each of the identified molecules is examined. Briefly, bone marrow cells from mice is generated as described. The wild type and KO cells are then cultured in media alone (negative control), 100 nm EF2505, 10 μg/ml NTHi, or 1 μg/ml LPS (positive control) for 24 h. Concentrations of IL-6 and TNFα (pro-inflammatory cytokines that are known to be produced in response to NF-kB activation; antibodies from PharMingen) released into the cultured supernatants will be measured by enzyme-linked immunosorbent assay (ELISA). NF-kB signaling is critical to most host-protective antimicrobial responses (Hayden et al., 2006). It is activated downstream of many PRRs (Hayden et al., 2006; Hacker and Karin, 2006). NF-kB function in the respiratory epithelium is likely also critical to EF2505-induced StIR.

### Example 7

### Develop an Effective Stimulant of Lung Innate Resistance to Bioterror Pathogens Based on EF2505 in Combination with Synthetic TLR Agonists

The present invention demonstrated the principle of broadly and effectively stimulating innate resistance of the lungs. EF2505 appears to stimulate multiple innate immune pathways, and displays an efficacy against both Gram+ and Gram− organisms. Supplementation of EF2505 with stimulation of additional innate receptors (Trinchieri and Sher, 2007) may be desirable. EF2505 would be useful against the Class A bioterror pathogens Bacillus anthracis, Yersinia pestis, and Francisella tularensis.

### Example 8

### Efficacy of EF2505 in Stimulating Innate Resistance of the Lungs to Bioterror Pathogens

EF2505 aerosolized at 1.0 mg/ml for 20 minutes protects mice effectively against S. pneumoniae and P. aeruginosa.

Pretreatment with aerosolized EF2505 is performed exactly as described (Clement et al., 2008). Challenge with aerosolized S. pneumoniae is performed exactly as described (Clement et al., 2008). For the bioterror pathogens, the dose is targeted to 5×LD₅₀. Ames strain B. anthracis spores, Y. pestis CO92, and F. tularensis Schu 4 are usually delivered by nasal instillation (Comer et al., 2006; Bielinska et al., 2007). Mice are anesthetized by intraperitoneal injection of ketamine (48 mg/kg) and xylazine (9.6 mg/kg) and then suspended vertically by their upper incisors. At this point, 20 ml of pathogen suspension is instilled into each naris, followed by 10 ml of PBS into each naris. For all pathogens, bacterial concentrations in nebulizers, nasal instillate, and lung tissue are determined by plating serial dilutions. The use of purified EF2505 that does not contain non-efficacious molecules could provide protection at a substantially lower protein concentration.

### Example 9

### Efficacy of Synthetic Ligands in Stimulating Innate Resistance of the Lungs to Bioterror Pathogens

EF2505 is predicted to activate LRR-containing innate receptors. The protective activity of EF2505 can be augmented by combination with innate receptor ligands for pathways that are not effectively activated by EF2505. Multiple synthetic ligands are available, including some that are FDA-approved such as imiquimod (a TLR7/8 agonist by Graceway Pharmaceuticals) and CpG 10101 (a TLR9 agonist by Coley Pharmaceuticals), (PHAD, or phosphorylated hexaacyl disaccharide, a TLR4 agonist by Avanti Polar Lipids) and Pam2CSK4 (a diacylated lipopeptide TLR2/6 agonist by InvivoGen). E. coli endotoxin (a TLR4 agonist) provides moderate resistance to S. pneumoniae (Clement et al., 2008). Pam2CSK4 at 10 μg/ml increases total bronchoalveolar lavage leukocytes four-fold. Similar studies with ODN 2395, imiquimod, MDP and lipid A.

### REFERENCES

The following references, to the extent that they provide exemplary procedural or other details supplementary to those set forth herein, are specifically incorporated herein by reference.
U.S. Pat. No. 4,554,101
U.S. Pat. No. 4,668,218
U.S. Pat. No. 5,458,135
U.S. Pat. No. 6,488,953
U.S. Pat. No. 6,737,045
U.S. Pat. No. 6,794,357
U.S. Pat. No. 6,797,258
U.S. patent Ser. No. 10/844,933
Abuchowski et al., J. Biol. Chem., 252:582, 1977.
Akinbi et al., J. Immunol., 165(10):5760-5766, 2000.
Bals and Hiemstra, Curr. Drug Targets, 7(6):743-750, 2006.
Bals and Hiemstra, Eur. Respir. J., 23(2):327-333, 2004
Barker et al., J. Med. Chem., 35:2040-2048, 1992.
Beauchamp et al., Anal. Biochem., 131:25, 1983.
Bielinska et al., Infect. Immun., 75(8):4020-4029, 2007.
Bouzianas, Expert Rev. Anti. Infect. Ther., 5(4):665-684, 2007.
Chen et al., Biochim. Biophy. Acta, 660:293, 1981.
Clement et al., Am. J. Respir. Crit. Care Med., 177(12):1322-1330, 2008.
Comer et al., J. Toxicol. Environ. Health, 69(11):1083-1095, 2006.
Dennis et al., JAMA, 285:2763-2773, 2001.
EP 0237507
Evans et al., Am. J. Respir. Cell Mol. Biol., 31(4):382-394, 2004.
Evans et al., Am. J. Respir. Cell Mol. Biol., 32(6):490-497, 2005.
Forteza et al., Am. J. Respir. Cell Mol. Biol., 32(5):462-469, 2005.
Hacker and Karin, Sci. STKE, 2006(357):rel3, 20, 2006.
Hassani et al., Clin. Immunol., 111(1):1-15, 2004.
Hayden et al., Oncogene, 25(51):6758-6780, 2006.
Hruby et al., Biochem J., 268:249-262, 1990.
Janeway, Jr. and Medzhitov, Annu. Rev. Immunol., 20:197-216, 2002.
Kita et al., Drug Des. Delivery, 6:157, 1990.
Knauf et al., J. Biol. Chem., 263:15064, 1988.
Knowles et al., J. Clin. Invest., 109(5):571-577, 2002.
Kyte and Doolittle, J. Mol. Biol., 157(1):105-132, 1982.
Martin and Frevert, Proc. Am. Thorac. Soc., 2(5):403-411, 2005.
Medzhitov and Janeway, Jr., Curr. Opin. Immunol., 9(1):4-9, 1997.
Morgan and Gainor, Ann. Rep. Med. Chem., 24:243-252, 1989.
Or et al., J. Org. Chem., 56:3146-3149, 1991.
PCT Appln. PCT/US04/14887
PCT Appln. WO 94/06498
PCT Appln. WO 94/08552
PCT Appln. WO 94/16970
PCT Appln. WO 97/25086
PCT Appln. WO 98/35888
Physicians' Desk Reference
Rogan et al., Respir. Res., 7:29, 2006.
Schmitt et al., Arch. Intern. Med., 167(7):655-662, 2007.
Travis et al., Curr. Opin. Immunol., 13(1):89-95, 2001.
Trinchieri and Sher, Nat. Rev. Immunol., 7(3):179-190, 2007.
Tsutsumi et al., J. Controlled Rel., 33:447, 1995.
Williams et al., Am. J. Respir. Cell Mol. Biol., 34(5):527-536. 10, 2006.

What is claimed is:

1. A method of attenuating a microbial infection comprising administering an effective amount of a polypeptide comprising at least 50 contiguous amino acids of an Enterococcus faecalis protein EF2505 (SEQ ID NO:2) to an individual that has or is at risk of developing such an infection.

2. The method of claim 1, wherein the polypeptide comprises at least amino acids 111 to 449 of SEQ ID NO:2

3. The method of claim 1, wherein the polypeptide comprises at most amino acids 28 to amino acid 449 of SEQ ID NO:2.

4. The method of claim 1, wherein the subject has been exposed to a pathogenic microbe.

5. The method of claim 1, wherein the microbe is a virus, a bacteria, or a fungus.

6. The method of claim 5, wherein the virus is Adenoviridae, Coronaviridae, Filoviridae, Flaviviridae, Hepadnaviridae, Herpesviridae, Orthomyxoviridae, Paramyxovirinae, Pneumovirinae, Picornaviridae, Poxyiridae, Retroviridae, Togaviridae, Parainfluenza, Influenza, H5N1, Marburg, Ebola, Severe acute respiratory syndrome coronavirus, Yellow fever, Human respiratory syncytial, Hantavirus, or Vaccinia virus.

7. The method of claim 5, wherein the bacteria is Bacillus anthracis, Yersinia pestis, Francisella tularensis, Pseudomonas aerugenosa or Staphylococcus aureas.

8. The method of claim 5, wherein the fungus is a Aspergillus, Candida, Cryptococcus, Histoplasma, Coccidioides, Blastomyces, Zygometes, or Pneumocystis.

9. The method of claim 1, wherein the protein or segment or derivative thereof is administered in a nebulized formulation.

10. The method of claim 1, wherein the protein or segment of derivative thereof is administered in an amount of from about 0.1 mg/kg to about 100 mg/kg of the individual's body weight.

11. The method of claim 1, wherein the polypeptide, is at least 75% identical to a sequence of SEQ ID NO:2.

12. The method of claim 1, wherein the polypeptide, is at least 80% identical to a sequence of SEQ ID NO:2.

13. The method of claim 1, wherein the polypeptide, is at least 85% identical to a sequence of SEQ ID NO:2.

14. The method of claim 1, wherein the polypeptide, is at least 90% identical to a sequence of SEQ ID NO:2.

15. The method of claim 1, wherein the polypeptide, is at least 95% identical to a sequence of SEQ ID NO:2.

16. The method of claim 1, wherein the polypeptide is identical to a sequence of SEQ ID NO:2.

17. A pharmaceutically acceptable composition comprising an Enterococcus faecalis protein EF2505 or segment or derivative thereof, an anti-inflammatory agent, and one or more pharmaceutical excipients.

18. The composition of claim 17, wherein the EF2505 protein or segment or derivative thereof is at least 75% identical to a sequence of SEQ ID NO:2.

19. The composition of claim 17, wherein the EF2505 protein or segment or derivative thereof is at least 80% identical to a sequence of SEQ ID NO:2.

20. The composition of claim 17, wherein EF2505 the protein or segment or derivative thereof is at least 85% identical to a sequence of SEQ ID NO:2.

21. The composition of claim 17, wherein the EF2505 protein or segment or derivative thereof is at least 90% identical to a sequence of SEQ ID NO:2.

22. The composition of claim 17, wherein the EF2505 protein or segment or derivative thereof is at least 95% identical to a sequence of SEQ ID NO:2.

23. The composition of claim 17, wherein the EF2505 protein or segment or derivative thereof is identical to a sequence of SEQ ID NO:2.

24. A method of identifying compounds that stimulate mammalian innate immune resistance to pathogens, comprising the steps of: measuring the binding of a compound to leucine-rich repeat containing proteins to determine a compound which exhibits binding; and measuring the ability of compounds which bind to leucine-rich repeat containing proteins to activate the mammalian immune system, wherein a compound that binds to leucine-rich repeat containing proteins and activate the mammalian immune system is a compound that stimulates mammalian innate immune resistance to pathogens.

25. The method of claim 24, wherein said compound is in a bacterial lysate.

26. The method of claim 24, wherein said compound is a recombinant bacterial protein.

27. The method of claim 24, wherein said leucine-rich repeat containing proteins are small leucine rich repeat proteoglycans.

28. The method of claim 24, wherein said leucine-rich repeat containing proteins are Toll-like receptors.

29. The method of claim 24, wherein said activation of the mammalian immune system is measured by activation of NF-kB.

30. A compound that binds to leucine-rich repeat containing proteins and activates the mammalian immune system to stimulate mammalian innate immune resistance to pathogens, wherein said compound is identified using the method of claim 24.
